(19) United States
(12) Patent Application Publication
(10) Pub. No.: US 2025/0388680 A1
(43) Pub. Date: Dec. 25, 2025

(54) CHIMERIC RECEPTORS AND METHODS OF USE THEREOF

(71) Applicant: Kite Pharma, Inc., Santa Monica, CA (US)

(72) Inventors: Nicole Cannon, Santa Monica, CA (US); Nikitha Gandra, Hayward, CA (US); Samuel T. Haile, Castro Valley, CA (US); Edward H. Liao, Petaluma, CA (US); Richard Smith, Belmont, CA (US); Sean C. Yoder, Simi Valley, CA (US)

(21) Appl. No.: 19/197,496

(22) Filed: May 2, 2025

Related U.S. Application Data
(60) Provisional application No. 63/642,573, filed on May 3, 2024.

(51) Int. Cl. C07K 16/28; A61K 35/17; A61K 40/11; A61K 40/31; A61K 40/42; A61P 35/00; A61P 37/04; C07K 14/705; C07K 14/725; C12N 5/0783

(52) U.S. Cl. CPC C07K 16/2851; A61K 35/17; A61K 40/11; A61K 40/31; A61K 40/4224; A61P 35/00; A61P 37/04; C07K 14/7051; C07K 14/70517; C07K 14/70521; C12N 5/0636; A61K 2239/13; A61K 2239/17; A61K 2239/21; A61K 2239/22; C07K 2317/565; C07K 2317/622; C12N 2510/00

(57) ABSTRACT

Provided herein are antibodies and antigen binding fragments thereof targeting the human C-type lectin-like molecule-1 (CLL-1) protein, as well as chimeric antigen receptors (CAR) derived from such antibodies and fragments, and the uses thereof.

### CROSS-REFERENCE TO RELATED APPLICATIONS

This application claims priority to U.S. Provisional Application No. 63/642,573, filed May 3, 2024, which is incorporated herein in its entirety for all purposes.

### SEQUENCE LISTING

The instant application contains a Sequence Listing which has been submitted electronically in XML format and is hereby incorporated by reference in its entirety. Said XML copy, created on Apr. 25, 2025, is named K-1162-US-NP_SL.xml and is 73,297 bytes in size.

### FIELD

This disclosure relates to antibodies and antigen binding fragments thereof targeting the human C-type lectin-like molecule-1 (CLL-1) protein, as well as chimeric antigen receptors (CAR) derived from such antibodies and fragments, and the uses thereof.

### BACKGROUND

C-type lectin-like-1 (CLL-1, also known as CLEC-1, CLEC12A, MICL, Dendritic Cell-Associated Lectin-1 (DCAL-1), and DCAL-2) is a glycoprotein receptor and member of a family of C-type lectin-like receptors involved in the regulation of cell proliferation and immune regulation. CLL-1 is expressed in hematopoietic cells, primarily on innate immune cells including monocytes, granulocytes, dendritic cells, as well as myeloid progenitor cells. CLL-1 has been implicated in the regulation of myeloid cell proliferation and differentiation and is present on acute myeloid (myelogenous) leukemia (AML) cells as well as on leukemic stem cells.

Accordingly, targeting CLL-1 can be useful in treating multiple diseases, including but not limited to, acute myeloid (myelogenous) leukemia (AML), chronic myeloid (myelogenous) leukemia (CML), chronic myelomonocytic leukemia (CMML), juvenile myelomonocytic leukemia, atypical chronic myeloid leukemia, acute promyelocytic leukemia (APL), acute monocytic leukemia, acute monoblastic leukemia, acute erythroid leukemia, acute megakaryoblastic leukemia, myelodysplastic syndrome (MDS), myeloproliferative disorder, myeloid neoplasm, myeloid sarcoma), Blastic Plasmacytoid Dendritic Cell Neoplasm (BPDCN).

CLL-1 may additionally play a role in inflammatory or autoimmune diseases such as rheumatoid arthritis, psoriasis, allergies, asthma, Crohn's disease, IBD, IBS, fibromyalgia, mastocytosis, and Celiac disease.

Engineered immune cells have been shown to possess desired qualities in therapeutic treatments, particularly in oncology. Two main types of engineered immune cells are those that contain chimeric antigen receptors (termed “CARs” or “CAR-Ts”) and T-cell receptors (“TCRs”). These engineered cells are engineered to endow them with antigen specificity while retaining or enhancing their ability to recognize and kill a target cell. Chimeric antigen receptors may comprise, for example, (i) an antigen-specific component (“antigen binding molecule”), (ii) transmembrane domains, and (iii) one or more activating domains. Each domain may be heterogeneous, that is, comprised of sequences derived from (or corresponding to) different protein chains. Chimeric antigen receptor-expressing immune cells (such as T cells) may be used in various therapies, including cancer therapies. It will be appreciated that one or more costimulatory domains may be used to enhance the activation of CAR-expressing cells against target antigens, and therefore increase the potency of adoptive immunotherapy.

### SUMMARY

Provided herein are antibodies and antigen binding fragments thereof targeting the human C-type lectin-like molecule-1 (CLL-1) protein, as well as chimeric antigen receptors (CAR) derived from such antibodies and fragments, and the uses thereof.

One embodiment of the present disclosure provides an antibody or antigen-binding fragment thereof having specificity to the human C-type lectin-like molecule-1 (CLL-1) protein, comprising a heavy chain variable region (VH) comprising a VH CDR1, a VH CDR2 and a VH CDR3, and a light chain variable region (VL) comprising a VL CDR1, a VL CDR2 and a VL CDR3, wherein the VH CDR1, VH CDR2, VH CDR3, VL CDR1, VL CDR2 and VL CDR3 comprise, respectively, (i) the VH CDR1, VH CDR2 and VH CDR3 sequences of SEQ ID NO:2 and the VL CDR1, VL CDR2 and VL CDR3 sequences of SEQ ID NO:1; (ii) the VH CDR1, VH CDR2 and VH CDR3 sequences of SEQ ID NO:15 and the VL CDR1, VL CDR2 and VL CDR3 sequences of SEQ ID NO:14; (iii) the VH CDR1, VH CDR2 and VH CDR3 sequences of SEQ ID NO:24 and the VL CDR1, VL CDR2 and VL CDR3 sequences of SEQ ID NO:23; (iv) the VH CDR1, VH CDR2 and VH CDR3 sequences of SEQ ID NO:34 and the VL CDR1, VL CDR2 and VL CDR3 sequences of SEQ ID NO:33; (v) the VH CDR1, VH CDR2 and VH CDR3 sequences of SEQ ID NO:46 and the VL CDR1, VL CDR2 and VL CDR3 sequences of SEQ ID NO:45; or (vi) the VH CDR1, VH CDR2 and VH CDR3 sequences of SEQ ID NO:55 and the VL CDR1, VL CDR2 and VL CDR3 sequences of SEQ ID NO:54.

In some embodiments, the VH CDR1, VH CDR2, VH CDR3, VL CDR1, VL CDR2 and VL CDR3 comprise, respectively, the sequences of (i) SEQ ID NOs: 6, 7, 8, 3, 4 and 5; (ii) SEQ ID NOs: 18, 19, 20, 16, 4 and 17; (iii) SEQ ID NOs: 28, 29, 30, 25, 26 and 27; (iv) SEQ ID NOs: 38, 39, 40, 35, 36 and 37; (v) SEQ ID NOs: 49, 50, 51, 47, 4 and 48; or (vi) SEQ ID NOs: 59, 60, 61, 56, 57 and 58.

In some embodiments, the VH CDR1, VH CDR2, VH CDR3, VL CDR1, VL CDR2 and VL CDR3 comprise, respectively, the sequences of SEQ ID NOs: 6, 7, 8, 3, 4 and 5. In some embodiments, the VH comprises the sequence of SEQ ID NO:2 and the VL comprises the sequence of SEQ ID NO:1.

In some embodiments, the VH CDR1, VH CDR2, VH CDR3, VL CDR1, VL CDR2 and VL CDR3 comprise, respectively, the sequences of SEQ ID NOs: 18, 19, 20, 16, 4 and 17. In some embodiments, the VH comprises the sequence of SEQ ID NO:15 and the VL comprises the sequence of SEQ ID NO:14.

In some embodiments, the VH CDR1, VH CDR2, VH CDR3, VL CDR1, VL CDR2 and VL CDR3 comprise, respectively, the sequences of SEQ ID NOs: 28, 29, 30, 25, 26 and 27. In some embodiments, the VH comprises the sequence of SEQ ID NO:24 and the VL comprises the sequence of SEQ ID NO:23.

In some embodiments, the VH CDR1, VH CDR2, VH CDR3, VL CDR1, VL CDR2 and VL CDR3 comprise, respectively, the sequences of SEQ ID NOs: 38, 39, 40, 35, 36 and 37. In some embodiments, the VH comprises the sequence of SEQ ID NO:34 and the VL comprises the sequence of SEQ ID NO:33.

In some embodiments, the VH CDR1, VH CDR2, VH CDR3, VL CDR1, VL CDR2 and VL CDR3 comprise, respectively, the sequences of SEQ ID NOs: 49, 50, 51, 47, 4 and 48. In some embodiments, the VH comprises the sequence of SEQ ID NO:46 and the VL comprises the sequence of SEQ ID NO:45.

In some embodiments, the VH CDR1, VH CDR2, VH CDR3, VL CDR1, VL CDR2 and VL CDR3 comprise, respectively, the sequences of SEQ ID NOs: 59, 60, 61, 56, 57 and 58. In some embodiments, the VH comprises the sequence of SEQ ID NO:55 and the VL comprises the sequence of SEQ ID NO:54.

Also provided, in one embodiment, is a chimeric antigen receptor (CAR) having specificity to the human CLL-1 protein, comprising an antigen-binding fragment of the present disclosure, a transmembrane domain, and an intracellular activating domain.

In some embodiments, the antigen-binding fragment is a single chain fragment (scFv).

In some embodiments, the transmembrane domain is a transmembrane domain of 4-1BB, an alpha chain of a T cell receptor, a beta chain of a T cell receptor, a zeta chain of a T cell receptor, CD3 epsilon, CD4, CD5, CD8 alpha, CD9, CD16, CD19, CD22, CD28, CD33, CD37, CD45, CD64, CD80, CD86, CD134, CD137, or CD154. In some embodiments, the transmembrane domain is a CD8 alpha transmembrane domain or a CD28 transmembrane domain. In some embodiments, the CD8 alpha transmembrane domain comprises the sequence of SEQ ID NO: 77, or wherein the CD28 transmembrane domain comprises the sequence of SEQ ID NO: 70.

In some embodiments, the intracellular activation domain is a signaling domain derived from CD3 zeta, CD3 epsilon, CD3 delta, and CD3 gamma. In some embodiments, the intracellular activation domain is CD3 zeta signaling domain.

In some embodiments, the CD3 zeta signaling domain comprises the sequence of SEQ ID NO: 68.

In some embodiments, the CAR further comprises a costimulatory domain. In some embodiments, the costimulatory domain is a signaling domain of 2B4, 4-1BB, B7-H3, BAFFR, BLAME, BTLA, CD100, CD103, CD11a, CD11b, CD11c, CD11d, CD150, CD160, CD18, CD19, CD19a, CD2, CD247, CD27, CD276, CD28, CD29, CD3 delta, CD3 epsilon, CD3 gamma, CD30, CD4, CD40, CD49a, CD49D, CD49f, CD69, CD7, CD84, CD8alpha, CD8beta, CD96, CDS, CEACAM1, CRT AM, cytokine receptor, DAP-10, DNAM1, Fc gamma receptor, GADS, GITR, HVEM, IA4, ICAM-1, ICOS, Ig alpha, IL-2R beta, IL-2R gamma, IL-7R alpha, integrin, IPO-3, ITGA4, ITGA4, ITGA6, ITGAD, ITGAE, ITGAL, ITGAM, ITGAX, ITGB1, ITGB2, ITGB7, KIRDS2, LAT, LFA-1, LIGHT, LTBR, Ly108, Ly9, MHC class 1 molecule, NKG2C, NKG2D, NKp30, NKp44, NKp46, NKp80, OX-40, PAG, PD-1, PSGL1, SELPLG, SLAMF4, SLAMF6, SLAMF7, SLP-76, TNFR2, TNFSFi4, TRANCE, VLA1, VLA-6, a TNF receptor protein, a ligand to CD83, a Toll ligand receptor, an activating NK cell receptor, or an immunoglobulin. In some embodiments, the costimulatory domain is a CD28 costimulatory domain. In some embodiments, the CD28 costimulatory domain comprises the sequence of SEQ ID NO: 67.

In some embodiments, the CAR further comprises a hinge domain. In some embodiments, the hinge domain is a hinge domain of IgG1, IgG2, IgG3, IgG4, IgA, IgD, IgE, IgM, CD28, or CD8 alpha. In some embodiments, the hinge domain is a hinge domain of CD8 alpha comprising the sequence of SEQ ID NO: 66. In some embodiments, the hinge domain is a truncated IgG4 hinge domain comprising the sequence of SEQ ID NO: 69. In some embodiments, the hinge domain is a hinge domain of CD28 comprising the sequence of SEQ ID NO: 71.

In some embodiments, the CAR comprises a sequence selected from the group consisting of SEQ ID NOs: 10, 12, 13, 22, 32, 42, 44, 53, and 63.

Also provided, in one embodiment, is a nucleic acid encoding the antibody or antigen-binding fragment thereof, or the CAR of the present disclosure. Also provided is a vector comprising the nucleic acid.

Still further provided is a composition comprising the antibody or antigen-binding fragment thereof or the CAR of the present disclosure, and a pharmaceutically acceptable carrier.

Still further provided is a cell encoding or expressing the antibody or antigen-binding fragment thereof or the CAR of the present disclosure, optionally wherein the cell is an immune cell, optionally wherein the cell is a T cell. In some embodiments, the T cell is a CD8+ T cell or CD4+ T cell.

Another embodiment provides a method of generating an engineered cell, the method comprising transfecting or transducing a cell with a nucleic acid or a vector of the present disclosure.

In another embodiment, a method of treating cancer or an autoimmune disease in a subject in need thereof is provided, the method comprising administering to the subject one or more cells that encode or comprise the antibody or antigen-binding fragment thereof or the CAR of the present disclosure.

Still further provided is a method of inducing an immune response in a subject, the method comprising administering to the subject one or more cells that encode or comprise the antibody or antigen-binding fragment thereof or the CAR of the present disclosure.

In some embodiments, the cells are CD8+ T cells or CD4+ T cells.

In some embodiments, the cancer is acute lymphoblastic leukemia (ALL), acute myeloid leukemia (AML), B cell prolymphocytic leukemia, B cell acute lymphoid leukemia (BALL), blastic plasmacytoid dendritic cell neoplasm, Burkitt's lymphoma, chronic lymphocytic leukemia (CLL), chronic myelogenous leukemia (CML), chronic myelomonocytic leukemia (CMML), juvenile myelomonocytic leukemia, chronic myeloid leukemia, chronic or acute leukemia, acute promyelocytic leukemia (APL), acute monoblastic leukemia, acute erythroid leukemia, acute megakaryoblastic leukemia, diffuse large B cell lymphoma (DLBCL), follicular lymphoma (FL), hairy cell leukemia, Hodgkin's Disease, malignant lymphoproliferative conditions, MALT lymphoma, Marginal zone lymphoma, monoclonal gammapathy of undetermined significance (MGUS), multiple myeloma, myeloproliferative disorder, myeloid neoplasm, myeloid sarcoma, myelodysplasia and myelodysplastic syndrome (MDS), non-Hodgkin's lymphoma (NHL), plasma cell proliferative disorder, plasmablastic lymphoma, plasmacytoid dendritic cell neoplasm, plasmacytomas, POEMS syndrome, primary mediastinal large B cell lymphoma (PMBC), small cell- or a large cell-follicular lymphoma, splenic marginal zone lymphoma (SMZL), systemic amyloid light chain amyloidosis, T cell acute lymphoid leukemia (TALL), T cell lymphoma, transformed follicular lymphoma, Waldenstrom macroglobulinemia, Mantle cell lymphoma (MCL), transformed follicular lymphoma (TFL), Primary mediastinal B cell lymphoma (PMBCL), blastic Plasmacytoid Dendritic Cell Neoplasm (BPDCN), Hairy cell lymphoma/leukemia, or a combination thereof.

In some embodiments, the autoimmune disease is selected from the group consisting of alopecia areata, autoimmune hemolytic anemia, autoimmune hepatitis, dermatomyositis, diabetes (type 1), celiac disease, autoimmune juvenile idiopathic arthritis, glomerulonephritis, Graves' disease, Guillain-Barré syndrome, idiopathic thrombocytopenic purpura, myasthenia gravis, autoimmune myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus erythematosus, autoimmune thyroiditis, Hashimoto's thyroiditis, autoimmune uveitis, vitiligo, and granulomatosis with polyangiitis (Wegener's).

In some embodiments, the one or more cells are allogeneic or autologous.

### BRIEF DESCRIPTION OF THE FIGURES

FIG. 1A and FIG. 1B show CAR expression in the cells from Donor 01. (FIG. 1A) Percentage of CARs positive in the CD3+ T cells; (FIG. 1B) Mean fluorescence intensity (MFI) of the CARs. Figures disclose “G4S” as SEQ ID NO: 74.

FIG. 1C and FIG. 1D show CAR expression in the cells from Donor 02. (FIG. 1C) Percentage of CARs positive in the CD3+ T cells; (FIG. 1D) Mean fluorescence intensity (MFI) of the CARs. Figures disclose “G4S” as SEQ ID NO: 74.

FIG. 1E and FIG. 1F show the vector copy number (VCN) of the constructed CARs in the cells from Donor 01 (FIG. 1E) and Donor 02 (FIG. 1F), respectively.

FIG. 2A and FIG. 2B show the CD4/CD8 ratio of memory T cells expressing the CARs in different donor, respectively.

FIG. 3A, FIG. 3B, and FIG. 3C show the percentage of Tscm, Tcm, Teff, and Temra in the memory T cells (CD3+, CD4+ or CD8+, respectively) from Donor 01 expressing the CARs.

FIG. 4A, FIG. 4B, and FIG. 4C show the percentage of Tscm, Tcm, Teff, and Temra in the memory T cells (CD3+, CD4+ or CD8+, respectively) from Donor 02 expressing the CARs.

FIGS. 5A-5F are graphs showing the in vitro cytotoxicity of CAR-T cells from Donor 01.

FIGS. 6A-6F are graphs showing the in vitro cytotoxicity of CAR-T cells from Donor 02.

FIG. 7A, FIG. 7B, FIG. 7C and FIG. 7D show the T cell activation by the CAR-T cells in vitro, as measured by release of IL-2 and INFgamma, respectively.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

It is to be further understood that wherever aspects are described herein with the language “comprising,” otherwise analogous aspects described in terms of “consisting of” and/or “consisting essentially of” are also provided.

Additionally, the terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% (i.e., ±10%). For example, about 3 mg can include any number between 2.7 mg and 3.3 mg (for 10%). Furthermore, particularly with respect to biological systems or processes, the terms can mean up to an order of magnitude or up to 5-fold of a value.

When particular values or compositions are provided in the application and claims, unless otherwise stated, the meaning of “about” or “comprising essentially of” should be assumed to be within an acceptable error range for that particular value or composition.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences.

The term “an equivalent nucleic acid or polynucleotide” refers to a nucleic acid having a nucleotide sequence having a certain degree of homology, or sequence identity, with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof. Likewise, “an equivalent polypeptide” refers to a polypeptide having a certain degree of homology, or sequence identity, with the amino acid sequence of a reference polypeptide. In some aspects, the sequence identity is at least about 70%, 75%, 80%, 85%, 90%, 95%, 98%, or 99%. In some aspects, the equivalent polypeptide or polynucleotide has one, two, three, four or five addition, deletion, substitution and their combinations thereof as compared to the reference polypeptide or polynucleotide. In some aspects, the equivalent sequence retains the activity (e.g., epitope-binding) or structure (e.g., salt-bridge) of the reference sequence.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that includes at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

A “single-chain variable fragment” or “scFv” refers to a fusion protein of the variable regions of the heavy (VH) and light chains (VL) of immunoglobulins. In some aspects, the regions are connected with a short linker peptide of ten to about 25 amino acids. The linker can be rich in glycine for flexibility, as well as serine or threonine for solubility, and can either connect the N-terminus of the VH with the C-terminus of the V_{L}, or vice versa. This protein retains the specificity of the original immunoglobulin, despite removal of the constant regions and the introduction of the linker. ScFv molecules are known in the art and are described, e.g., in U.S. Pat. No. 5,892,019.

The term antibody encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgG₅, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule includes two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments including either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

An “Fc” region includes two heavy chain fragments including the CH1 and CH2 domains of an antibody. The two heavy chain fragments are held together by two or more disulfide bonds and by hydrophobic interactions of the CH3 domains.

A “Fab fragment” includes one light chain and the CH1 and variable regions of one heavy chain. The heavy chain of a Fab molecule cannot form a disulfide bond with another heavy chain molecule. A “Fab fragment” includes one light chain and a portion of one heavy chain that contains the VH domain and the CH1 domain and also the region between the CH1 and CH2 domains, such that an interchain disulfide bond can be formed between the two heavy chains of two Fab′ fragments to form an F(ab′)₂ molecule. An “F(ab′)₂ fragment” contains two light chains and two heavy chains containing a portion of the constant region between the CH1 and CH2 domains, such that an interchain disulfide bond is formed between the two heavy chains. An F(ab′)₂ fragment thus is composed of two Fab′ fragments that are held together by a disulfide bond between the two heavy chains.

The “Fv region” includes the variable regions from both the heavy and light chains, but lacks the constant regions.

A “bivalent antigen binding molecule” includes two antigen binding sites. In some instances, the two binding sites have the same antigen specificities. Bivalent antigen binding molecules can be bispecific. A “multispecific antigen binding molecule” is one that targets more than one antigen or epitope. A “bispecific,” “dual-specific” or “bifunctional” antigen binding molecule is a hybrid antigen binding molecule or antibody, respectively, having two different antigen binding sites. The two binding sites of a bispecific antigen binding molecule will bind to two different epitopes, which can reside on the same or different protein targets.

Light chains are classified as either kappa or lambda (K, X). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VK) and heavy (VH) chain portions determine antigen recognition and specificity. Conversely, the constant domains of the light chain (CK) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region; the CH3 and CK domains actually include the carboxy-terminus of the heavy and light chain, respectively.

Antibodies disclosed herein may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks).

The heavy chain constant region of an antibody disclosed herein may be derived from different immunoglobulin molecules. For example, a heavy chain constant region of a polypeptide may include a CH1 domain derived from an IgG₁ molecule and a hinge region derived from an IgG₃ molecule. In another example, a heavy chain constant region can include a hinge region derived, in part, from an IgG₁ molecule and, in part, from an IgG₃ molecule. In another example, a heavy chain portion can include a chimeric hinge derived, in part, from an IgG₁ molecule and, in part, from an IgG₄ molecule.

As used herein, the term “light chain constant region” includes amino acid sequences derived from antibody light chain. Preferably, the light chain constant region includes at least one of a constant kappa domain or constant lambda domain.

As used herein, the term “hinge region” includes the portion of a heavy chain molecule that joins the CH1 domain to the CH2 domain. This hinge region includes approximately 25 residues and is flexible, thus allowing the two N-terminal antigen-binding regions to move independently. Hinge regions can be subdivided into three distinct domains: upper, middle, and lower hinge domains (Roux et al., J. Immunol 161:4083 (1998)).

As used herein, the term “chimeric antibody” will be held to mean any antibody wherein the immunoreactive region or site is obtained or derived from a first species and the constant region (which may be intact, partial or modified in accordance with the instant disclosure) is obtained from a second species. In certain embodiments the target binding region or site will be from a non-human source (e.g. mouse or primate) and the constant region is human.

As used herein, “percent humanization” is calculated by determining the number of framework amino acid differences (i.e., non-CDR difference) between the humanized domain and the germline domain, subtracting that number from the total number of amino acids, and then dividing that by the total number of amino acids and multiplying by 100.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

As indicated above, the variable region allows the antibody to selectively recognize and specifically bind epitopes on antigens. That is, the VK domain and VH domain, or subset of the complementarity determining regions (CDRs), of an antibody combine to form the variable region that defines a three-dimensional antigen-binding site. This quaternary antibody structure forms the antigen-binding site present at the end of each arm of the Y. More specifically, the antigen-binding site is defined by three CDRs on each of the VH and VK chains (i.e. CDR-H1, CDR-H2, CDR-H3, CDR-L1, CDR-L2 and CDR-L3). In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

The variable regions typically exhibit the same general structure of relatively conserved framework regions (FR) joined by the 3 hypervariable regions (i.e., “CDRs”). The CDRs from the two chains of each pair typically are aligned by the framework regions, which can enable binding to a specific epitope. From N-terminal to C-terminal, both light and heavy chain variable regions typically include the domains FR1, CDR1, FR2, CDR2, FR3, CDR3 and FR4. By convention, CDR regions in the heavy chain are typically referred to as HC CDR1, CDR2, and CDR3. The CDR regions in the light chain are typically referred to as LC CDR1, CDR2, and CDR3. The assignment of amino acids to each domain is typically in accordance with the definitions of Kabat, Chothia, or the AbM definition.

A number of definitions of the CDRs are commonly in use: Kabat numbering, Chothia numbering, AbM numbering, or contact numbering. The AbM definition is a compromise between the two used by Oxford Molecular's AbM antibody modelling software. The contact definition is based on an analysis of the available complex crystal structures.

CDR Numbering

| Loop | Kabat | AbM | Chothia | Contact |
|---|---|---|---|---|
| L1 | L24--L34 | L24--L34 | L24--L34 | L30--L36 |
| L2 | L50--L56 | L50--L56 | L50--L56 | L46--L55 |
| L3 | L89--L97 | L89--L97 | L89--L97 | L89--L96 |
| H1 | H31--H35B | H26--H35B | H26-- | H30--H35B |
| | (Kabat Numbering) | | H32 . . . 34 | |
| H1 | H31--H35 | H26--H35 | H26--H32 | H30--H35 |
| | (Chothia Numbering) | | | |
| H2 | H50--H65 | H50--H58 | H52--H56 | H47--H58 |
| H3 | H95--H102 | H95--H102 | H95--H102 | H93--H101 |

The CDR3 of the light chain and, particularly, the CDR3 of the heavy chain may constitute the most important determinants in antigen binding within the light and heavy chain variable regions. In some antibody constructs, the heavy chain CDR3 appears to constitute the major area of contact between the antigen and the antibody. In vitro selection schemes in which CDR3 alone is varied can be used to vary the binding properties of an antibody or determine which residues contribute to the binding of an antigen. Hence, CDR3 is typically the greatest source of molecular diversity within the antibody-binding site. H3, for example, can be as short as two amino acid residues or greater than 26 amino acids.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off} can be determined by techniques known to one of ordinary skill in the art, such as BIAcore® or KinExA.

The term “neutralizing” refers to an antigen binding molecule, scFv, or antibody, respectively, that binds to a ligand and prevents or reduces the biological effect of that ligand. This can be done, for example, by directly blocking a binding site on the ligand or by binding to the ligand and altering the ligand's ability to bind through indirect means (such as structural or energetic alterations in the ligand). In some embodiments, the term can also denote an antigen binding molecule that prevents the protein to which it is bound from performing a biological function.

The term “compete” when used in the context of antigen binding molecules that compete for the same epitope means competition between antigen binding molecules as determined by an assay in which the antigen binding molecule (e.g., antibody or immunologically functional fragment thereof) being tested prevents or inhibits (e.g., reduces) specific binding of a reference antigen binding molecule to an antigen. Numerous types of competitive binding assays can be used to determine if one antigen binding molecule competes with another, for example: solid phase direct or indirect radioimmunoassay (RIA), solid phase direct or indirect enzyme immunoassay (EIA), sandwich competition assay (Stahli et al., 1983, Methods in Enzymology 9:242-253); solid phase direct biotin-avidin EIA (Kirkland et al., 1986, J. Immunol. 137:3614-3619), solid phase direct labeled assay, solid phase direct labeled sandwich assay (Harlow and Lane, 1988, Antibodies, A Laboratory Manual, Cold Spring Harbor Press); solid phase direct label RIA using 1-125 label (Morel et al., 1988, Molec. Immunol. 25:7-15); solid phase direct biotin-avidin EIA (Cheung, et al., 1990, Virology 176:546-552); and direct labeled RIA (Moldenhauer et al., 1990, Scand. J. Immunol. 32:77-82).

As used herein, the term “epitope” refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain embodiments, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giegé R. et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303). Antibody:antigen crystals may be studied using well known X-ray diffraction techniques and may be refined using computer software such as X-PLOR (Yale University, 1992, distributed by Molecular Simulations, Inc.; see e.g. Meth Enzymol (1985) volumes 114 & 115, eds Wyckoff H W et al.; U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta Crystallogr D Biol Crystallogr 56(Pt 10): 1316-1323). Mutagenesis mapping studies may be accomplished using any method known to one of skill in the art. See, e.g., Champe M et al., (1995) J Biol Chem 270: 1388-1394 and Cunningham B C & Wells J A (1989) Science 244: 1081-1085 for a description of mutagenesis techniques, including alanine scanning mutagenesis techniques.

A “variant” of a polypeptide (e.g., an antigen binding molecule, or an antibody) includes an amino acid sequence wherein one or more amino acid residues are inserted into, deleted from and/or substituted into the amino acid sequence relative to another polypeptide sequence. Variants include fusion proteins.

A “therapeutically effective amount,” “effective dose,” “effective amount,” or “therapeutically effective dosage” of a therapeutic agent, e.g., engineered CAR T cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

### Chimeric Antigen Receptors (CAR)

Chimeric antigen receptors (CARs) and engineered T cell receptors (TCRs) are genetically engineered receptors. These engineered receptors can be readily inserted into and expressed by immune cells, such as T cells. A CAR can recognize a specific antigen and, when bound to that antigen, activate the immune cell to attack and destroy the cell bearing that antigen. When these antigens exist on tumor cells, an immune cell that expresses the CAR can target and kill the tumor cell.

CARs can be engineered to bind to an antigen (such as a cell-surface antigen) by incorporating an antigen binding molecule that interacts with that targeted antigen. Preferably, the antigen binding molecule is an antibody fragment thereof, and more preferably one or more single chain antibody fragment (“scFv”). An scFv is a single chain antibody fragment having the variable regions of the heavy and light chains of an antibody linked together. It will be appreciated that the antigen binding molecule is typically contained within the extracellular portion of the CAR such that it is capable of recognizing and binding to the antigen of interest. Bispecific and multispecific CARs are contemplated within the scope of the disclosure, with specificity to more than one target of interest.

An example CAR includes, in addition to the antigen binding molecule, a transmembrane domain and an activation domain. In some embodiments, the CAR can further include an extracellular domain between the antigen binding molecule and the transmembrane domain. The extracellular domain may include a hinge domain. In some embodiments, the CAR can further include a co-stimulatory domain.

### A. Antigen Binding Molecule

The antigen binding portion of the CAR of the instant disclosure can be an antibody or antigen binding fragment (in particular a scFv) targeting human CLL-1 (e.g., SEQ ID NO: 65, additional sequence information is contained in the CLL-1 Uniprot No: Q5QGZ9, as well as NCBI Reference Sequence NP_612210.4), such as those disclosed in Example 1.

Example anti-CLL-1 antibodies include those murine ones listed in Table 3 as in Example 1 (e.g., mAb_049, mAb_017, mAb_023, mAb_038, mAb_018, mAb_042). Also included are those that include the same CDRs as illustrated herein. In some embodiments, the disclosed antibodies and fragments include those that bind to the same epitope as those illustrated here, and those that compete with the instantly disclosed in binding to CLL-1.

In one embodiment, the VH CDRs are those VH CDRs in any one of the heavy chain variable regions selected from SEQ ID NOs: 2, 15, 24, 34, 46, and 55, or a variant thereof having one, two, or three deletions, additions, substitutions or the combinations thereof.

In one embodiment, the VL CDRs are those VL CDRs in any one of the light chain variable regions selected from SEQ ID NOs: 1, 14, 23, 33, 45, and 54, or a variant thereof having one, two, or three deletions, additions, substitutions or the combinations thereof.

In one embodiment, the VH CDR1 has a sequence according to any one of SEQ ID NOs: 6, 18, 28, 38, 49 and 59, or a variant thereof having one, two, or three deletions, additions, substitutions or the combinations thereof; the VH CDR2 has a sequence according to any one of SEQ ID NOs: 7, 19, 29, 39, 50 and 60, or a variant thereof having one, two, or three deletions, additions, substitutions or the combinations thereof; the VH CDR3 has a sequence according to any one of SEQ ID NOs: 8, 20, 30, 40, 51 and 61, or a variant thereof having one, two, or three deletions, additions, substitutions or the combinations thereof; the VL CDR1 has a sequence according to any one of SEQ ID NOs: 3, 16, 25, 35, 47 and 56, or a variant thereof having one, two, or three deletions, additions, substitutions or the combinations thereof; the VL CDR2 has a sequence according to any one of SEQ ID NOs: 4, 26, 36 and 57, or a variant thereof having one, two, or three deletions, additions, substitutions or the combinations thereof; and the VL CDR3 has a sequence according to any one of SEQ ID NOs: 5, 17, 27, 37, 48 and 58, or a variant thereof having one, two, or three deletions, additions, substitutions or the combinations thereof.

In one embodiment, the CDRs are those of mAb_049 as exemplified in Table 3. In one embodiment, the VH CDR1 includes the amino acid sequence of SEQ ID NO: 6 or a variant thereof having one, two, or three deletions, additions, substitutions or the combinations thereof, the VH CDR2 includes the amino acid sequence of SEQ ID NO: 7 or a variant thereof having one, two, or three deletions, additions, substitutions or the combinations thereof, the VH CDR3 includes the amino acid sequence of SEQ ID NO: 8 or a variant thereof having one, two, or three deletions, additions, substitutions or the combinations thereof, the VL CDR1 includes the amino acid sequence of SEQ ID NO: 3 or a variant thereof having one, two, or three deletions, additions, substitutions or the combinations thereof, the VL CDR2 includes the amino acid sequence of SEQ ID NO: 4 or a variant thereof having one, two, or three deletions, additions, substitutions or the combinations thereof, and the VL CDR3 includes the amino acid sequence of SEQ ID NO: 5 or a variant thereof having one, two, or three deletions, additions, substitutions or the combinations thereof.

In one embodiment, the CDRs are those of mAb_017 as exemplified in Table 3. In one embodiment, the VH CDR1 includes the amino acid sequence of SEQ ID NO: 18 or a variant thereof having one, two, or three deletions, additions, substitutions or the combinations thereof, the VH CDR2 includes the amino acid sequence of SEQ ID NO: 19 or a variant thereof having one, two, or three deletions, additions, substitutions or the combinations thereof, the VH CDR3 includes the amino acid sequence of SEQ ID NO: 20 or a variant thereof having one, two, or three deletions, additions, substitutions or the combinations thereof, the VL CDR1 includes the amino acid sequence of SEQ ID NO: 19 or a variant thereof having one, two, or three deletions, additions, substitutions or the combinations thereof, the VL CDR2 includes the amino acid sequence of SEQ ID NO: 4 or a variant thereof having one, two, or three deletions, additions, substitutions or the combinations thereof, and the VL CDR3 includes the amino acid sequence of SEQ ID NO: 17 or a variant thereof having one, two, or three deletions, additions, substitutions or the combinations thereof.

In one embodiment, the CDRs are those of mAb_023 as exemplified in Table 3. In one embodiment, the VH CDR1 includes the amino acid sequence of SEQ ID NO: 28 or a variant thereof having one, two, or three deletions, additions, substitutions or the combinations thereof, the VH CDR2 includes the amino acid sequence of SEQ ID NO: 29 or a variant thereof having one, two, or three deletions, additions, substitutions or the combinations thereof, the VH CDR3 includes the amino acid sequence of SEQ ID NO: 30 or a variant thereof having one, two, or three deletions, additions, substitutions or the combinations thereof, the VL CDR1 includes the amino acid sequence of SEQ ID NO: 25 or a variant thereof having one, two, or three deletions, additions, substitutions or the combinations thereof, the VL CDR2 includes the amino acid sequence of SEQ ID NO: 26 or a variant thereof having one, two, or three deletions, additions, substitutions or the combinations thereof, and the VL CDR3 includes the amino acid sequence of SEQ ID NO: 27 or a variant thereof having one, two, or three deletions, additions, substitutions or the combinations thereof.

In one embodiment, the CDRs are those of mAb_038 as exemplified in Table 3. In one embodiment, the VH CDR1 includes the amino acid sequence of SEQ ID NO: 38 or a variant thereof having one, two, or three deletions, additions, substitutions or the combinations thereof, the VH CDR2 includes the amino acid sequence of SEQ ID NO: 39 or a variant thereof having one, two, or three deletions, additions, substitutions or the combinations thereof, the VH CDR3 includes the amino acid sequence of SEQ ID NO: 40 or a variant thereof having one, two, or three deletions, additions, substitutions or the combinations thereof, the VL CDR1 includes the amino acid sequence of SEQ ID NO: 35 or a variant thereof having one, two, or three deletions, additions, substitutions or the combinations thereof, the VL CDR2 includes the amino acid sequence of SEQ ID NO: 36 or a variant thereof having one, two, or three deletions, additions, substitutions or the combinations thereof, and the VL CDR3 includes the amino acid sequence of SEQ ID NO: 37 or a variant thereof having one, two, or three deletions, additions, substitutions or the combinations thereof.

In one embodiment, the CDRs are those of mAb_018 as exemplified in Table 3. In one embodiment, the VH CDR1 includes the amino acid sequence of SEQ ID NO: 49 or a variant thereof having one, two, or three deletions, additions, substitutions or the combinations thereof, the VH CDR2 includes the amino acid sequence of SEQ ID NO: 50 or a variant thereof having one, two, or three deletions, additions, substitutions or the combinations thereof, the VH CDR3 includes the amino acid sequence of SEQ ID NO: 51 or a variant thereof having one, two, or three deletions, additions, substitutions or the combinations thereof, the VL CDR1 includes the amino acid sequence of SEQ ID NO: 47 or a variant thereof having one, two, or three deletions, additions, substitutions or the combinations thereof, the VL CDR2 includes the amino acid sequence of SEQ ID NO: 4 or a variant thereof having one, two, or three deletions, additions, substitutions or the combinations thereof, and the VL CDR3 includes the amino acid sequence of SEQ ID NO: 48 or a variant thereof having one, two, or three deletions, additions, substitutions or the combinations thereof.

In one embodiment, the CDRs are those of mAb_042 as exemplified in Table 3. In one embodiment, the VH CDR1 includes the amino acid sequence of SEQ ID NO: 59 or a variant thereof having one, two, or three deletions, additions, substitutions or the combinations thereof, the VH CDR2 includes the amino acid sequence of SEQ ID NO: 60 or a variant thereof having one, two, or three deletions, additions, substitutions or the combinations thereof, the VH CDR3 includes the amino acid sequence of SEQ ID NO: 61 or a variant thereof having one, two, or three deletions, additions, substitutions or the combinations thereof, the VL CDR1 includes the amino acid sequence of SEQ ID NO: 56 or a variant thereof having one, two, or three deletions, additions, substitutions or the combinations thereof, the VL CDR2 includes the amino acid sequence of SEQ ID NO: 57 or a variant thereof having one, two, or three deletions, additions, substitutions or the combinations thereof, and the VL CDR3 includes the amino acid sequence of SEQ ID NO: 58 or a variant thereof having one, two, or three deletions, additions, substitutions or the combinations thereof.

Also provided, in some embodiments, are those that include the same CDRs as any one of mAb_049, mAb_017, mAb_023, mAb_038, mAb_018, mAb_042 or its humanized counterparts. In some embodiments, the disclosed antibodies and fragments include those that bind to the same epitope as any one of mAb_049, mAb_017, mAb_023, mAb_038, mAb_018, mAb_042, or its humanized counterparts, and those that compete with any of them in binding to CLL-1.

It is appreciated that CDRs can be modified to include those having one, two or three amino acid addition, deletion and/or substitutions. In some embodiments, the substitutions can be conservative substitutions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

In some embodiments, the heavy chain variable region includes an amino acid sequence selected from the group consisting of SEQ ID NO: 2, SEQ ID NO: 15, SEQ ID NO: 24, SEQ ID NO: 34, SEQ ID NO: 46, or SEQ ID NO: 55, or a peptide having at least 80% (or at least 90%, 95%, 96%, 97%, 98%, 99%) sequence identity thereof while retaining the same CDRs.

In some embodiments, the light chain variable region includes an amino acid sequence selected from the group consisting of SEQ ID NO: 1, SEQ ID NO: 14, SEQ ID NO: 23, SEQ ID NO: 33, SEQ ID NO: 45, or SEQ ID NO: 54, or a peptide having at least 80% (or at least 90%, 95%, 96%, 97%, 98%, 99%) sequence identity thereof while retaining the same CDRs.

In some embodiments, the antibody or antigen binding fragment thereof provided herein includes a heavy chain variable domain including an amino acid sequence of SEQ ID NO: 2, or a peptide having at least 80% (or at least 90%, 95%, 96%, 97%, 98%, 99%) sequence identity thereof while retaining the same CDRs, and a light chain variable domain including an amino acid sequence of SEQ ID NO: 1, or a peptide having at least 80% (or at least 90%, 95%, 96%, 97%, 98%, 99%) sequence identity thereof while retaining the same CDRs.

In some embodiments, the antibody or antigen binding fragment thereof provided herein includes a heavy chain variable domain including an amino acid sequence of SEQ ID NO: 15, or a peptide having at least 80% (or at least 90%, 95%, 96%, 97%, 98%, 99%) sequence identity thereof while retaining the same CDRs, and a light chain variable domain including an amino acid sequence of SEQ ID NO: 14, or a peptide having at least 80% (or at least 90%, 95%, 96%, 97%, 98%, 99%) sequence identity thereof while retaining the same CDRs.

In some embodiments, the antibody or antigen binding fragment thereof provided herein includes a heavy chain variable domain including an amino acid sequence of SEQ ID NO: 24, or a peptide having at least 80% (or at least 90%, 95%, 96%, 97%, 98%, 99%) sequence identity thereof while retaining the same CDRs, and a light chain variable domain including an amino acid sequence of SEQ ID NO: 23, or a peptide having at least 80% (or at least 90%, 95%, 96%, 97%, 98%, 99%) sequence identity thereof while retaining the same CDRs.

In some embodiments, the antibody or antigen binding fragment thereof provided herein includes a heavy chain variable domain including an amino acid sequence of SEQ ID NO: 34, or a peptide having at least 80% (or at least 90%, 95%, 96%, 97%, 98%, 99%) sequence identity thereof while retaining the same CDRs, and a light chain variable domain including an amino acid sequence of SEQ ID NO: 33, or a peptide having at least 80% (or at least 90%, 95%, 96%, 97%, 98%, 99%) sequence identity thereof while retaining the same CDRs.

In some embodiments, the antibody or antigen binding fragment thereof provided herein includes a heavy chain variable domain including an amino acid sequence of SEQ ID NO: 46, or a peptide having at least 80% (or at least 90%, 95%, 96%, 97%, 98%, 99%) sequence identity thereof while retaining the same CDRs, and a light chain variable domain including an amino acid sequence of SEQ ID NO: 45, or a peptide having at least 80% (or at least 90%, 95%, 96%, 97%, 98%, 99%) sequence identity thereof while retaining the same CDRs.

In some embodiments, the antibody or antigen binding fragment thereof provided herein includes a heavy chain variable domain including an amino acid sequence of SEQ ID NO: 55 or a peptide having at least 80% (or at least 90%, 95%, 96%, 97%, 98%, 99%) sequence identity thereof while retaining the same CDRs, and a light chain variable domain including an amino acid sequence of SEQ ID NO: 54 or a peptide having at least 80% (or at least 90%, 95%, 96%, 97%, 98%, 99%) sequence identity thereof while retaining the same CDRs.

It will also be understood by one of ordinary skill in the art that antibodies as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, 95%, 98%, or 99% identical to the starting sequence.

The disclosure further provides for pharmaceutical compositions including an antigen binding molecule to CLL-1 together with a pharmaceutically acceptable diluent, carrier, solubilizer, emulsifier, preservative and/or adjuvant. In certain embodiments, pharmaceutical compositions will include more than one different antigen binding molecule to CLL-1. In certain embodiments, pharmaceutical compositions will include more than one antigen binding molecule to CLL-1 wherein the antigen binding molecules to CLL-1 bind more than one epitope. In some embodiments, the various antigen binding molecules will not compete with one another for binding to CLL-1.

In other embodiments, the pharmaceutical composition can be selected for parenteral delivery, for inhalation, or for delivery through the digestive tract, such as orally. The preparation of such pharmaceutically acceptable compositions is within the ability of one skilled in the art. In certain embodiments, buffers are used to maintain the composition at physiological pH or at a slightly lower pH, typically within a pH range of from about 5 to about 8. In certain embodiments, when parenteral administration is contemplated, a therapeutic composition can be in the form of a pyrogen-free, parenterally acceptable aqueous solution including a desired antigen binding molecule to CLL-1, with or without additional therapeutic agents, in a pharmaceutically acceptable vehicle. In certain embodiments, a vehicle for parenteral injection is sterile distilled water in which an antigen binding molecule to CLL-1, with or without at least one additional therapeutic agent, is formulated as a sterile, isotonic solution, properly preserved. In certain embodiments, the preparation can involve the formulation of the desired molecule with polymeric compounds (such as polylactic acid or polyglycolic acid), beads or liposomes that can provide for the controlled or sustained release of the product which can then be delivered via a depot injection. In certain embodiments, implantable drug delivery devices can be used to introduce the desired molecule.

In some embodiments, the antigen binding molecule is used as a diagnostic or validation tool. The antigen binding molecule can be used to assay the amount of CLL-1 present in a sample and/or subject. In some embodiments, the diagnostic antigen binding molecule is not neutralizing. In some embodiments, the antigen binding molecules disclosed herein are used or provided in an assay kit and/or method for the detection of CLL-1 in mammalian tissues or cells in order to screen/diagnose for a disease or disorder associated with changes in levels of CLL-1. The kit can include an antigen binding molecule that binds CLL-1, along with means for indicating the binding of the antigen binding molecule with CLL-1, if present, and optionally CLL-1 protein levels.

An antibody or antigen binding molecule is said to “specifically bind” its target antigen when the dissociation constant (K_{d}) is ˜1×10⁻⁷ M. The antigen binding molecule specifically binds antigen with “high affinity” when the K_{d} is 1-5×10⁻⁹ M, and with “very high affinity” when the K_{d} is 1-5×10⁻¹⁰ M. In one embodiment, the antigen binding molecule has a K_{d} of 10⁻⁹ M. In one embodiment, the off-rate is <1×10⁻⁵. In other embodiments, the antigen binding molecules will bind to human CLL-1 with a K_{d} of between about 10⁻⁷ M and 10⁻¹³ M, and in yet another embodiment the antigen binding molecules will bind with a K_{d} 1.0-5×10⁻¹⁰.

In some embodiments, the antibody or antigen binding molecules of the present disclosure specifically bind CLL-1 (e.g., hCLL-1). In certain embodiments, an anti-CLL-1 antibody or antigen binding molecule of the present disclosure binds human CLL-1 with a K_{D} of less than 1×10⁻⁶ M, less than 1×10⁻⁷ M, less than 1×10⁻⁸ M, or less than 1×10⁻⁹ M. In one particular embodiment, the anti-CLL-1 antibody or antigen binding molecules binds human CLL-1 with a K_{D} of less than 1×10⁻⁷ M. In another embodiment, the anti-CLL-1 antibody or antigen binding molecules binds human CLL-1 with a K_{D} of less than 1×10⁻⁸ M. In some embodiments, the anti-CLL-1 antibody or antigen binding molecules binds human CLL-1 with a K_{D} of about 1×10⁻⁷ M, about 2×10⁻⁷ M, about 3×10⁻⁷ M, about 4×10⁻⁷ M, about 5×10⁻⁷ M, about 6×10⁻⁷ M, about 7×10⁻⁷ M, about 8×10⁻⁷ M, about 9×10⁻⁷ M, about 1×10⁻⁸ M, about 2×10⁻⁸ M, about 3×10⁻⁸ M, about 4×10⁻⁸ M, about 5×10⁻⁸ M, about 6×10⁻⁸ M, about 7×10⁻⁸ M, about 8×10⁻⁸ M, about 9×10⁻⁸ M, about 1×10⁻⁹ M, about 2×10⁻⁹ M, about 3×10⁻⁹ M, about 4×10⁻⁹ M, about 5×10⁻⁹ M, about 6×10⁻⁹ M, about 7×10⁻⁹ M, about 8×10⁻⁹ M, about 9×10⁻⁹ M, about 1×10⁻¹⁰ M, or about 5×10⁻¹⁰ M. In certain embodiments, the K_{D} is calculated as the quotient of k_{off}/kₒₙ, and the kₒₙ and k_{off} are determined using a monovalent antibody, such as a Fab fragment, as measured by, e.g., BIAcore® surface plasmon resonance technology. In other embodiments, the K_{D} is calculated as the quotient of k_{off}/kₒₙ, and the kₒₙ and k_{off} are determined using a bivalent antibody, such as a Fab fragment, as measured by, e.g., BIAcore® surface plasmon resonance technology.

In other embodiments, the anti-CLL-1 antibody or antigen binding molecule binds human CLL-1-Fc with a K_{D} of less than 1×10⁻⁹ M, less than 3×10⁻⁹ M, less than 5×10⁻⁹ M, less than 1×10⁻¹⁰ M, less than 3×10⁻¹⁰ M, or less than 5×10⁻¹⁰ M. In other embodiments, the anti-CLL-1 antibody or antigen binding molecules binds cyno CLL-1-Fc with a K_{D} of less than 1×10⁻⁵ M, less than 1×10⁻⁶ M, less than 1×10⁻⁷ M, less than 1×10⁻⁸ M, less than 1×10⁻⁹ M, or less than 1×10⁻¹⁰ M.

In some embodiments, the anti-CLL-1 antibody or antigen binding molecule binds human CLL-1 with an association rate (k_{off}) of less than 1×10⁻⁴ M⁻¹ s⁻¹, less than 2×10⁻⁴ M⁻¹ s⁻¹, less than 3×10⁻⁴ M⁻¹ s⁻¹, less than 4×10⁻⁴ M⁻¹ s⁻¹, less than 5×10⁻⁴ M⁻¹ s⁻¹, less than 6×10⁻⁴ M⁻¹ s⁻¹, less than 7×10⁻⁴ M⁻¹ s⁻¹, less than 8×10⁻⁴ M⁻¹ s⁻¹, less than 9×10⁴ M⁻¹ s⁻¹, less than 1×10⁻⁵ M⁻¹ s⁻¹, less than 2×10⁻⁵ M⁻¹ s⁻¹, less than 3×10⁻⁵ M⁻¹ s⁻¹, less than 4×10⁻⁵ M⁻¹ s⁻¹, less than 5×10⁻⁵ M⁻¹ s⁻¹, less than 6×10⁻⁵ M⁻¹ s⁻¹, less than 7×10⁻⁵ M⁻¹ s⁻¹, less than 8×10⁻⁵ M⁻¹ s⁻¹, less than 9×10⁻⁵ M⁻¹ s⁻¹, less than 1×10⁻⁶ M⁻¹ s⁻¹, less than 2×10⁻⁶ M⁻¹ s⁻¹, less than 3×10⁻⁶ M-s⁻¹, less than 4×10⁻⁶ M⁻¹ s⁻¹, less than 5×10⁻⁶ M⁻¹ s⁻¹, less than 6×10⁻⁶ M⁻¹ s⁻¹, less than 7×10⁻⁶ M⁻¹ s⁻¹, less than 8×10⁻⁶ M⁻¹ s⁻¹, less than 9×10⁻⁶ M⁻¹ s⁻¹, or less than 1×10⁻⁷ M⁻¹ s⁻¹. In certain embodiments, the kₒₙ is determined using a monovalent antibody, such as a Fab fragment, as measured by, e.g., BIAcore® surface plasmon resonance technology. In other embodiments, the kₒₙ is determined using a bivalent antibody as measured by, e.g., BIAcore® surface plasmon resonance technology.

In some embodiments, the anti-CLL-1 antibody or antigen binding molecule binds human CLL-1 with an dissociation rate (k_{off}) of less than 1×10⁻² s⁻¹, less than 2×10⁻² s⁻¹, less than 3×10⁻² s⁻¹, less than 4×10⁻² s⁻¹, less than 5×10⁻² s⁻¹, less than 6×10⁻² s⁻¹, less than 7×10⁻² s⁻¹, less than 8×10⁻² s⁻¹, less than 9×10⁻² s⁻¹, less than 1×10⁻³ s⁻¹, less than 2×10⁻³ s⁻¹, less than 3×10⁻³ s⁻¹, less than 4×10⁻³ s⁻¹, less than 5×10⁻³ s⁻¹, less than 6×10⁻³ s⁻¹, less than 7×10⁻³ s⁻¹, less than 8×10⁻³ s⁻¹, less than 9×10⁻³ s⁻¹, less than 1×10⁻⁴ s⁻¹, less than 2×10⁻⁴ s⁻¹, less than 3×10⁻⁴ s⁻¹, less than 4×10⁻⁴ s⁻¹, less than 5×10⁴ s⁻¹, less than 6×10⁻⁴ s⁻¹, less than 7×10⁻⁴ s⁻¹, less than 8×10⁻⁴ s⁻¹, less than 9×10⁻⁴ s⁻¹, less than 1×10⁻⁴ s⁻¹, or less than 5×10⁻⁴ s⁻¹ In certain embodiments, the k_{off} is determined using a monovalent antibody, such as a Fab fragment, as measured by, e.g., BIAcore® surface plasmon resonance technology. In other embodiments, the k_{off} is determined using a bivalent antibody as measured by, e.g., BIAcore® surface plasmon resonance technology.

In some embodiments, the heavy chain variable region and the light chain variable region are in a single polypeptide. In some embodiments, the antibody or antigen binding fragment thereof provided herein is a scFv.

For a scFv, in some embodiments, the VH is disposed N-terminal to the VL, optionally through a linker. In some embodiments, the VH is disposed C-terminal to the VL, optionally through a linker.

### B. Extracellular Domain and Transmembrane Domain

Chimeric antigen receptors include an extracellular domain. Extracellular domains often include a hinge portion, sometimes referred to as the “spacer” region. A variety of hinges can be employed in accordance with the disclosure, including portions or derivatives of the molecules as listed above. In certain embodiments, the hinge portion is a hinge region of IgG1, IgG2, IgG3, IgG4, IgA, IgD, IgE, IgM, CD28, or CD8 alpha, any truncation thereof, or any combination thereof.

In certain embodiments, the hinge region includes an amino acid sequence that is at least about 75%, at least about 80%, at least about 85%, at least about 90%, at least about 95%, at least about 96%, at least about 97%, at least about 98%, at least about 99%, or 100% identical to the extracellular domain amino acid sequences set forth herein.

In certain embodiments, the hinge region is a hinge region of CD8 alpha, which includes an amino acid sequence of SEQ ID NO: 66, and sequences having at least 80%, 85%, 90%, 95%, 98% or 99% sequence identity to SEQ ID NO: 66.

In certain embodiments, the hinge region is a hinge region of IgG4(S108P) short, which includes an amino acid sequence of SEQ ID NO: 69, and sequences having at least 80%, 85%, 90%, 95%, 98% or 99% sequence identity to SEQ ID NO: 69.

In certain embodiments, the hinge region is a hinge region of CD28, which includes an amino acid sequence of SEQ ID NO: 71, and sequences having at least 80%, 85%, 90%, 95%, 98% or 99% sequence identity to SEQ ID NO: 71.

The CAR can be designed with a transmembrane domain. In some instances, the transmembrane domain can be selected or modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein.

Non-limiting examples of such proteins include CD28, CD8alpha, CD8beta, 4-1BB, B7-H3, BAFFR, BLAME, BTLA, CD100, CD103, CD11a, CD11b, CD11c, CD11d, CD160, CD18, CD19, CD19a, CD2, CD247, CD27, CD276, CD29, CD3 delta, CD3 epsilon, CD3 gamma, CD30, CD4, CD40, CD49a, CD49D, CD49f, CD69, CD7, CD79a, CD84, CD96, CDS, CEACAM1, CRT AM, DAP-10, DNAM1, Fc gamma receptor, GADS, GITR, HVEM, IA4, ICAM-1, ICOS, IL-2R beta, IL-2R gamma, IL-7R alpha, ITGA4, ITGA6, ITGAD, ITGAE, ITGAL, ITGAM, ITGAX, ITGB1, ITGB2, ITGB7, KIRDS2, LAT, LFA-1, LIGHT, LTBR, Ly9, NKG2C, NKG2D, NKp30, NKp44, NKp46, NKp80, OX-40, PAG, PD-1, PSGL1, SELPLG, SLAM, SLAMF4, SLAMF6, SLAMF7, SLP-76, TNFR2, TNFSFi4, TRANCE, VLA1, VLA-6, a cytokine receptor, a MHC class 1 molecule, a SLAM protein, a TNF receptor protein, a Toll ligand receptor, an activating NK cell receptor, an immunoglobulin protein, and an integrin.

In a particular example, the transmembrane domain is one from CD8a. Example sequences of CD28 transmembrane domain include SEQ ID NO:77 and sequences having at least 80%, 85%, 90%, 95%, 98% or 99% sequence identity to SEQ ID NO:77.

In a particular example, the transmembrane domain is one from CD28. Example sequences of CD28 transmembrane domain include SEQ ID NO:70 and sequences having at least 80%, 85%, 90%, 95%, 98% or 99% sequence identity to SEQ ID NO:70.

### C. Intracellular Domain—Costimulatory Domain and Activation Domain

The intracellular (cytoplasmic) domain of the engineered T cells of the disclosure can provide activation of at least one of the normal effector functions of the immune cell. Effector function of a T cell, for example, may refer to cytolytic activity or helper activity, including the secretion of cytokines. The intracellular domain may include at least an activation domain. The intracellular domain can also include one or more costimulatory domains.

### Costimulatory Domains

A “costimulatory domain” as used herein refers to a molecule that provides a signal which mediates a T cell response, including, but not limited to, proliferation, activation, differentiation, and the like. Costimulatory domain can provide a signal in addition to the primary signal provided by an activating molecule as described herein.

Chimeric antigen receptors may incorporate costimulatory (signaling) domains to increase their potency. For example, CD28 is a costimulatory protein found naturally on T-cells.

A variety of costimulatory molecules are set forth herein, but it will be appreciated that additional costimulatory molecules are also included within the scope of this disclosure.

It will be appreciated that suitable costimulatory domains within the scope of the disclosure include the signaling domain (or other suitable portion) of 2B4, 4-1BB, B7-H3, BAFFR, BLAME, BTLA, CD100, CD103, CD11a, CD11b, CD11c, CD11d, CD150, CD160, CD18, CD19, CD19a, CD2, CD247, CD27, CD276, CD28, CD29, CD3 delta, CD3 epsilon, CD3 gamma, CD30, CD4, CD40, CD49a, CD49D, CD49f, CD69, CD7, CD84, CD8alpha, CD8beta, CD96, CDS, CEACAM1, CRT AM, cytokine receptor, DAP-10, DNAM1, Fc gamma receptor, GADS, GITR, HVEM, IA4, ICAM-1, ICOS, Ig alpha, IL-2R beta, IL-2R gamma, IL-7R alpha, integrin, IPO-3, ITGA4, ITGA4, ITGA6, ITGAD, ITGAE, ITGAL, ITGAM, ITGAX, ITGB1, ITGB2, ITGB7, KIRDS2, LAT, LFA-1, LIGHT, LTBR, Ly108, Ly9, MHC class 1 molecule, NKG2C, NKG2D, NKp30, NKp44, NKp46, NKp80, OX-40, PAG, PD-1, PSGL1, SELPLG, SLAMF4, SLAMF6, SLAMF7, SLP-76, TNFR2, TNFSFi4, TRANCE, VLA1, VLA-6, a TNF receptor protein, a ligand to CD83, a Toll ligand receptor, an activating NK cell receptor, or an immunoglobulin. It will be appreciated that additional costimulatory molecules, or fragments thereof, not listed above are within the scope of the disclosure.

In a particular example, the costimulatory domain is one from CD28. Example sequences of CD28 transmembrane domain include SEQ ID NO:67 and sequences having at least 80%, 85%, 90%, 95%, 98% or 99% sequence identity to SEQ ID NO: 67.

### Activating Domains

An “activation domain” refers to a molecule on a T cell, e.g., the TCR/CD3 complex that specifically binds with a cognate stimulatory ligand present on an antigen present cell. Suitable activating molecules are described herein. The activation domain is part of the intracellular (cytoplasmic) portion of a CAR. The intracellular domain of a CAR can provide activation of at least one of the normal effector functions of the immune cell. Effector function of a T cell, for example, may refer to cytolytic activity or helper activity, including the secretion of cytokines.

CD3 is an element of the T cell receptor on native T cells, and has been shown to be an important intracellular activating element in CARs. In a preferred embodiment, the CD3 is CD3 zeta, CD3 epsilon, CD3 delta, or CD3 gamma.

A commonly used activation domain is the CD3zeta domain by itself or combined with any other desired intracellular domain(s). CD3 is an element of the T cell receptor on native T cells, and has been shown to be an important intracellular activating element in CARs. Example sequences of CD3zeta include SEQ ID NO:68 and sequences having at least 80%, 85%, 90%, 95%, 98% or 99% sequence identity to SEQ ID NO:68.

### Domain Orientation

In some embodiments, an antigen binding system of the present disclosure may include an antigen binding system that includes one or more, or all, of a leader peptide (P), an antigen binding motif (such as scFv) (B), a hinge domain (H), a transmembrane domain (T), a costimulatory domain (C), and an activation domain (A). In some instances, an antigen binding system is configured according to the following: B H T A. In some instances, an antigen binding system is configured according to the following: P B H T A. In some instances, an antigen binding system is configured according to the following: B H T C A. In some instances, an antigen binding system is configured according to the following: P B H T C A. In some embodiments, the antigen binding system includes a VH and a VL, optionally wherein the CAR is configured according to the following: VH-VL-H-T-C-A, VL-VH-H-T-C-A, P-VH-VL-H-T-C-A or P-VL-VH-H-T-C-A. In some embodiments, the VH and the VL are connected by a linker (L), optionally wherein the CAR is configured according to the following, from N-terminus to C-terminus: VH-L-VL-H-T-C-A, VH-L-VL-H-T-C-A, P-VH-L-VL-H-T-C-A or P-VH-L-VL-H-T-C-A. In some embodiments, the binding motif is connected by a second linker (L′), optionally wherein the CAR is configured according to the following, from N-terminus to C-terminus: VH-L-VL-L′-H-T-C-A, VH-L-VL-L′-H-T-C-A, P-VH-L-VL-L′-H-T-C-A or P-VH-L-VL-L′-H-T-C-A. It will be appreciated that the antigen binding system typically further includes one or more costimulatory molecules as described herein.

In some instances, an antigen binding system is configured similar to a type II transmembrane domain (e.g., the extracellular domain is on the C-terminal end and the intracellular portions are on the N-terminal end). Accordingly, in some embodiments, the antigen binding system is configured according to the following: B H T A (from C-terminus to N-terminus). In some instances, an antigen binding system is configured according to the following: P B H T A (from C-terminus to N-terminus). In some instances, an antigen binding system is configured according to the following: B H T C A (from C-terminus to N-terminus). In some instances, an antigen binding system is configured according to the following: P B H T C A (from C-terminus to N-terminus). In some embodiments, the antigen binding system includes a VH and a VL, optionally wherein the CAR is configured according to the following: VH-VL-H-T-C-A, VL-VH-H-T-C-A, P-VH-VL-H-T-C-A or P-VL-VH-H-T-C-A (from C-terminus to N-terminus). In some embodiments, the VH and the VL are connected by a linker (L), optionally wherein the CAR is configured according to the following, from N-terminus to C-terminus: VH-L-VL-H-T-C-A, VH-L-VL-H-T-C-A, P-VH-L-VL-H-T-C-A or P-VH-L-VL-H-T-C-A (from C-terminus to N-terminus). In some embodiments, the binding motif is connected by a second linker (L′), optionally wherein the CAR is configured according to the following, from N-terminus to C-terminus: VH-L-VL-L′-H-T-C-A, VH-L-VL-L′-H-T-C-A, P-VH-L-VL-L′-H-T-C-A or P-VH-L-VL-L′-H-T-C-A (from C-terminus to N-terminus). It will be appreciated that the antigen binding system typically further includes one or more costimulatory molecules as described herein.

In certain embodiments, the leader peptide includes an amino acid sequence that is at least about 75%, at least about 80%, at least about 85%, at least about 90%, at least about 95%, at least about 96%, at least about 97%, at least about 98%, at least about 99%, or 100% identical to the amino acid sequence of SEQ ID NO: 64. In some embodiments, the leader peptide includes the amino acid sequence of SEQ ID NO: 64.

The domains such as the antigen binding motif, the extracellular domain (hinge domain), the transmembrane domain, the activation domain and/or the one or more co-stimulatory domains can be selected from any source described in the present disclosure. Exemplary CAR constructs in accordance with the disclosure are set forth in Table 4 as in Example 2.

Optionally, short linkers may form linkages between any or some of the extracellular, transmembrane, and intracellular domains of the CAR.

In some embodiments, the linker is a peptide linker. In some embodiments, the linker has a length of about four to about fifty amino acids. In certain embodiments, the linker is selected from the group consisting of (GS)n, (GGGS)n (SEQ ID NO: 72), (GGGGS)n (SEQ ID NO: 74), and (GSGGS)n (SEQ ID NO: 75). In some embodiments, the n is 0-8. In some embodiments, the linker includes an amino acid sequence of GGGGSGGGGSGGGGS (SEQ ID NO: 73). In some embodiments, the linker includes an amino acid sequence of GGGGSGGGGSGGGGSGGGGS (SEQ ID NO:76).

In one embodiment, the linker is a three amino acid peptide linker “AAA”.

In certain embodiments, the short linkers may form linkages between VH and VL in a scFv as the antigen binding motif. The linker can be used to link the VL to the VH, or the VH to the VL, depending on the orientation of the VL to VH. In certain embodiments, the linker is selected from the group consisting of SEQ ID NOs:72-76.

In some embodiments, the antigen binding motif includes an amino acid sequence selected from the group consisting of SEQ ID NO: 9, 11, 21, 31, 41, 43, 52 and 62, or a variant that is at least about 75%, at least about 80%, at least about 85%, at least about 90%, at least about 95%, at least about 96%, at least about 97%, at least about 98%, at least about 99%, or 100% identical to any of the amino acid sequence of SEQ ID NO: 9, 11, 21, 31, 41, 43, 52 and 62.

In some embodiments, the CAR construct includes an amino acid sequence selected from the group consisting of SEQ ID NO: 10, 12, 13, 22, 32, 42, 44, 53 and 63, or a variant that is at least about 75%, at least about 80%, at least about 85%, at least about 90%, at least about 95%, at least about 96%, at least about 97%, at least about 98%, at least about 99%, or 100% identical to any of the amino acid sequence of SEQ ID NO: 10, 12, 13, 22, 32, 42, 44, 53 and 63.

In some embodiments, isolated nucleic acids are provided including a promoter operably linked to a first polynucleotide encoding an antigen binding molecule, a hinge domain, a transmembrane domain, optionally at least one costimulatory molecule, and an activating domain. In some embodiments, the nucleic acid construct is contained within a viral vector. In some embodiments, the viral vector is selected from the group consisting of retroviral vectors, murine leukemia virus vectors, SFG vectors, adenoviral vectors, lentiviral vectors, adeno-associated virus (AAV) vectors, Herpes virus vectors, and vaccinia virus vectors. In some embodiments, the nucleic acid is contained within a plasmid.

### CAR-Immune Cells

Immune cells enclosing a CAR of the present disclosure or one or more polynucleotides encoding the CAR are also provided.

As described, an engineered receptors can be inserted into and expressed by an immune cell, such as T cell, which can recognize a specific antigen and, when bound to that antigen, activate the immune cell to attack and destroy the cell bearing that antigen.

Various types of immune cells have been tested for expressing CARs. Non-limiting examples include T cells, NK cells, macrophages and monocytes. A T cell, in some embodiment, may be an alpha beta T cell. In some embodiments, the T cell is a gamma delta T cell.

NK cells are a type of cytotoxic (cell toxic) lymphocyte that represent a major component of the inherent immune system. NK cells reject tumors and cells infected by viruses. It works through the process of apoptosis or programmed cell death. They were termed “natural killers” because they do not require activation in order to kill cells. T-cells play a major role in cell-mediated-immunity (no antibody involvement). Its T-cell receptors (TCR) differentiate themselves from other lymphocyte types. The thymus, a specialized organ of the immune system, is primarily responsible for the T cell's maturation. Helper T-cells (e.g., CD4+ cells), Cytotoxic T-cells (also known as TC, cytotoxic T lymphocyte, CTL, T-killer cell, cytolytic T cell, CD8+ T-cells or killer T cell), Memory T-cells ((i) stem memory T_{SCM} cells, like naive cells, are CD45RO−, CCR7+, CD45RA+, CD62L+(L-selectin), CD27+, CD28+ and IL-7Rα+, but they also express large amounts of CD95, IL-2Rβ, CXCR3, and LFA-1, and show numerous functional attributes distinctive of memory cells); (ii) central memory T_{CM} cells express L-selectin and the CCR7, they secrete IL-2, but not IFNγ or IL-4; (iii) effector memory T_{EM} cells, however, do not express L-selectin or CCR7 but produce effector cytokines like IFNγ and IL-4), Regulatory T-cells (Tregs, suppressor T cells, or CD4+CD25+regulatory T cells and (iv) T_{EMRA} cells, effector memory cells re-expressing CD45RA (“terminally differentiated cells”)), Natural Killer T-cells (NKT) and Gamma Delta T-cells. B-cells, on the other hand, play a principal role in humoral immunity (with antibody involvement). It makes antibodies and antigens and performs the role of antigen-presenting cells (APCs) and turns into memory B-cells after activation by antigen interaction. In mammals, immature B-cells are formed in the bone marrow, where its name is derived from.

In some embodiments, the immune cell is autologous, or allogeneic. Without limitation, an autologous immune cell is obtained from a patient and engineered to express a CAR. An allogeneic immune cell may be derived from donor cells, such as stem cells.

The present disclosure also provides polynucleotides or nucleic acid molecules encoding the CAR. In some embodiments, the polynucleotide is an mRNA molecule. In some embodiments, the mRNA can be introduced into a target cell for expressing the antibody or fragment thereof.

Methods of preparing the engineered immune cells are also provided. In some embodiments, the method entails transfecting or transducing an immune cell with a polynucleotide or vector encoding the CAR.

### Methods of Treatment

Using adoptive immunotherapy, native T cells can be (i) removed from a patient, (ii) genetically engineered to express a chimeric antigen receptor (CAR) that binds to at least one tumor antigen (iii) expanded ex vivo into a larger population of engineered T cells, and (iv) reintroduced into the patient. See e.g., U.S. Pat. Nos. 7,741,465, and 6,319,494. After the engineered T cells are reintroduced into the patient, they mediate an immune response against cells expressing the tumor antigen. See e.g., Krause et al., J. Exp. Med., Volume 188, No. 4, 1998 (619-626). This immune response includes secretion of IL-2 and other cytokines by T cells, the clonal expansion of T cells recognizing the tumor antigen, and T cell-mediated specific killing of target-positive cells. See Hombach et al., Journal of Immun. 167: 6123-6131 (2001).

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method including inducing, enhancing, suppressing or otherwise modifying an immune response. Examples of immunotherapy include, but are not limited to, T cell therapies. T cell therapy can include adoptive T cell therapy, tumor-infiltrating lymphocyte (TIL) immunotherapy, autologous cell therapy, engineered autologous cell therapy (eACT), and allogeneic T cell transplantation. However, one of skill in the art would recognize that the conditioning methods disclosed herein would enhance the effectiveness of any transplanted T cell therapy. Examples of T cell therapies are described in U.S. Patent Publication Nos. 2014/0154228 and 2002/0006409, U.S. Pat. No. 5,728,388, and International Publication No. WO 2008/081035.

The T cells of the immunotherapy can come from any source known in the art. For example, T cells can be differentiated in vitro from a hematopoietic stem cell population, or T cells can be obtained from a subject. T cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by references in its entirety.

The term “engineered Autologous Cell Therapy,” which can be abbreviated as “eACT™,” also known as adoptive cell transfer, is a process by which a patient's own T cells are collected and subsequently genetically altered to recognize and target one or more antigens expressed on the cell surface of one or more specific tumor cells or malignancies. T cells can be engineered to express, for example, chimeric antigen receptors (CAR) or T cell receptor (TCR). CAR positive (+) T cells are engineered to express an extracellular single chain variable fragment (scFv) with specificity for a particular tumor antigen linked to an intracellular signaling part including at least one activating domain and in certain embodiments, at least one costimulatory domain. The costimulatory domain can be derived from (or correspond to), e.g., CD28, and the activating domain can be derived from (or correspond to) e.g., CD3-zeta. In certain embodiments, the CAR is designed to have two, three, four, or more costimulatory domains.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient.

The term “allogeneic” refers to any material derived from one individual which is then introduced to another individual of the same species, e.g., allogeneic T cell transplantation.

In some aspects, the disclosure therefore includes a method for treating or preventing a condition associated with undesired and/or elevated CLL-1 levels in a patient, including administering to a patient in need thereof an effective amount of at least one isolated antigen binding molecule, CAR, or TCR disclosed herein.

### Cancer

Methods are provided for treating diseases or disorders, including cancer. In some embodiments, the disclosure relates to creating a T cell-mediated immune response in a subject, including administering an effective amount of the engineered immune cells of the present application to the subject. In some embodiments, the T cell-mediated immune response is directed against a target cell or cells. In some embodiments, the engineered immune cell includes a chimeric antigen receptor (CAR), or a T cell receptor (TCR). In some embodiments, the target cell is a tumor cell. In some aspects, the disclosure includes a method for treating or preventing a malignancy, said method including administering to a subject in need thereof an effective amount of at least one isolated antigen binding molecule described herein. In some aspects, the disclosure includes a method for treating or preventing a malignancy, said method including administering to a subject in need thereof an effective amount of at least one immune cell, wherein the immune cell includes at least one chimeric antigen receptor, T cell receptor, and/or isolated antigen binding molecule as described herein.

In some aspects, the disclosure includes a pharmaceutical composition including at least one antigen binding system, antibody, or antigen binding fragment thereof as described herein and a pharmaceutically acceptable excipient. In some embodiments, the pharmaceutical composition further includes an additional active agent.

The antigen binding molecules, CARs, TCRs, immune cells, and the like of the disclosure can be used to treat myeloid diseases including but not limited to acute myeloid leukemia (AML), chronic myelogenous leukemia (CML), chronic myelomonocytic leukemia (CMML), juvenile myelomonocytic leukemia, atypical chronic myeloid leukemia, acute promyelocytic leukemia (APL), acute monoblastic leukemia, acute erythroid leukemia, acute megakaryoblastic leukemia, myelodysplastic syndrome (MDS), myeloproliferative disorder, myeloid neoplasm, myeloid sarcoma), Blastic Plasmacytoid Dendritic Cell Neoplasm (BPDCN), or combinations thereof. Additional diseases include inflammatory and/or autoimmune diseases such as rheumatoid arthritis, psoriasis, allergies, asthma, Crohn's disease, IBD, IBS, fibromyalga, mastocytosis, and Celiac disease.

It will be appreciated that target doses for CAR⁺/CAR-T⁺/TCR⁺ cells can range from 1×10⁶-2×10¹⁰ cells/kg, preferably 2×10⁶ cells/kg, more preferably. It will be appreciated that doses above and below this range may be appropriate for certain subjects, and appropriate dose levels can be determined by the healthcare provider as needed. Additionally, multiple doses of cells can be provided in accordance with the disclosure.

Also provided are methods for reducing the size of a tumor in a subject, including administering to the subject an engineered cell of the present disclosure to the subject, wherein the cell includes a chimeric antigen receptor, a T cell receptor, or a T cell receptor based chimeric antigen receptor including an antigen binding molecule binds to an antigen on the tumor. In some embodiments, the subject has a solid tumor, or a blood malignancy such as lymphoma or leukemia. In some embodiments, the engineered cell is delivered to a tumor bed.

In some embodiments, the cancer is present in the bone marrow of the subject. In some embodiments, the engineered cells are autologous T cells. In some embodiments, the engineered cells are allogeneic T cells. In some embodiments, the engineered cells are heterologous T cells. In some embodiments, the engineered cells of the present application are transfected or transduced in vivo. In other embodiments, the engineered cells are transfected or transduced ex vivo. As used herein, the term “in vitro cell” refers to any cell which is cultured ex vivo. In particular, an in vitro cell can include a T cell.

The methods can further include administering one or more chemotherapeutic agent. In certain embodiments, the chemotherapeutic agent is a lymphodepleting (preconditioning) chemotherapeutic. Beneficial preconditioning treatment regimens, along with correlative beneficial biomarkers are described in U.S. Provisional Patent Applications 62/262,143 and 62/167,750 which are hereby incorporated by reference in their entirety herein. These describe, e.g., methods of conditioning a patient in need of a T cell therapy including administering to the patient specified beneficial doses of cyclophosphamide (between 200 mg/m²/day and 2000 mg/m²/day) and specified doses of fludarabine (between 20 mg/m²/day and 900 mg/m²/day). A preferred dose regimen involves treating a patient including administering daily to the patient about 500 mg/m²/day of cyclophosphamide and about 60 mg/m²/day of fludarabine for three days prior to administration of a therapeutically effective amount of engineered T cells to the patient.

In other embodiments, the antigen binding molecule, transduced (or otherwise engineered) cells (such as CARs or TCRs), and the chemotherapeutic agent are administered each in an amount effective to treat the disease or condition in the subject.

In certain embodiments, compositions including CAR-expressing immune effector cells disclosed herein may be administered in conjunction with any number of chemotherapeutic agents. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL™, Bristol-Myers Squibb) and doxetaxel (TAXOTERE®, Rhone-Poulenc Rorer); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS2000; difluoromethylomithine (DMFO); retinoic acid derivatives such as Targretin™ (bexarotene), Panretin™, (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Combinations of chemotherapeutic agents are also administered where appropriate, including, but not limited to CHOP, i.e., Cyclophosphamide (Cytoxan©), Doxorubicin (hydroxydoxorubicin), Vincristine (Oncovin©), and Prednisone.

In some embodiments, the chemotherapeutic agent is administered at the same time or within one week after the administration of the engineered cell, polypeptide, or nucleic acid. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the engineered cell, polypeptide, or nucleic acid. In other embodiments, the chemotherapeutic agent is administered at least 1 month before administering the cell, polypeptide, or nucleic acid. In some embodiments, the methods further include administering two or more chemotherapeutic agents.

A variety of additional therapeutic agents may be used in conjunction with the compositions described herein. For example, potentially useful additional therapeutic agents include PD-1 inhibitors such as nivolumab (Opdivo©), pembrolizumab (Keytruda©), pembrolizumab, pidilizumab, and atezolizumab.

Additional therapeutic agents suitable for use in combination with the disclosure include, but are not limited to, ibrutinib (Imbruvica©), ofatumumab (Arzerra©), rituximab (Rituxan*), bevacizumab (Avastin©), trastuzumab (Herceptin®), trastuzumab emtansine (KADCYLA®), imatinib (Gleevec®), cetuximab (Erbitux®), panitumumab (Vectibix®), catumaxomab, ibritumomab, ofatumumab, tositumomab, brentuximab, alemtuzumab, gemtuzumab, erlotinib, gefitinib, vandetanib, afatinib, lapatinib, neratinib, axitinib, masitinib, pazopanib, sunitinib, sorafenib, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimetinib, selumetinib, trametinib, binimetinib, alectinib, ceritinib, crizotinib, aflibercept, adipotide, denileukin diftitox, mTOR inhibitors such as Everolimus and Temsirolimus, hedgehog inhibitors such as sonidegib and vismodegib, CDK inhibitors such as CDK inhibitor (palbociclib).

In additional embodiments, the composition including CAR-containing immune can be administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium, Cox-2 inhibitors, and sialylates. Exemplary analgesics include acetaminophen, oxycodone, tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

In certain embodiments, the compositions described herein are administered in conjunction with a cytokine. “Cytokine” as used herein is meant to refer to proteins released by one cell population that act on another cell as intercellular mediators. Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

### Inflammatory Diseases and Autoimmune Diseases

Also provided are methods and uses for treating inflammatory conditions or autoimmune disease. In some embodiments, the inflammatory disease or condition to be treated by the disclosed antibodies, fragments and compositions includes one or more of Alzheimer's disease, Addison's disease, atherosclerosis, ankylosing spondylitis, arthritis, osteoarthritis (OA), rheumatoid arthritis (RA), psoriatic arthritis (PA), ankylosing spondylitis, asthma, atherosclerosis, chronic obstructive pulmonary disease (COPD), Crohn's disease, colitis, dermatitis, diverticulitis, fibromyalgia, hepatitis, irritable bowel syndrome (IBS), systemic lupus erythematous (SLE), nephritis, Parkinson's disease (PD), vasculitis, and ulcerative colitis.

In some embodiments, the autoimmune disease or condition to be treated by the disclosed antibodies, fragments and compositions includes one or more of alopecia areata, autoimmune hemolytic anemia, autoimmune hepatitis, dermatomyositis, diabetes (type 1), celiac disease, autoimmune juvenile idiopathic arthritis, glomerulonephritis, Graves' disease, Guillain-Barré syndrome, idiopathic thrombocytopenic purpura, myasthenia gravis, autoimmune myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus erythematosus, autoimmune thyroiditis, Hashimoto's thyroiditis, autoimmune uveitis, vitiligo, and granulomatosis with polyangiitis (Wegener's).

Rheumatoid arthritis (RA) is a long-term autoimmune disorder that primarily affects joints. It typically results in warm, swollen, and painful joints. Pain and stiffness often worsen following rest. Most commonly, the wrist and hands are involved, with the same joints typically involved on both sides of the body. The disease may also affect other parts of the body. While the cause of rheumatoid arthritis is not clear, it is believed to involve a combination of genetic and environmental factors. The underlying mechanism involves the body's immune system attacking the joints. This results in inflammation and thickening of the joint capsule. The goals of treatment are to reduce pain, decrease inflammation, and improve a person's overall functioning. Pain medications, steroids, and NSAIDs are frequently used to help with symptoms. A group of medications called disease-modifying antirheumatic drugs (DMARDs), such as hydroxychloroquine and methotrexate, may be used to try to slow the progression of disease.

Osteoarthritis (OA) is a type of joint disease that results from breakdown of joint cartilage and underlying bone. The most common symptoms are joint pain and stiffness. Initially, symptoms may occur only following exercise, but over time may become constant. Other symptoms may include joint swelling, decreased range of motion, and when the back is affected weakness or numbness of the arms and legs. Causes include previous joint injury, abnormal joint or limb development, and inherited factors. Risk is greater in those who are overweight, have one leg of a different length, and have jobs that result in high levels of joint stress. Osteoarthritis is believed to be caused by mechanical stress on the joint and low grade inflammatory processes. Treatment includes exercise, efforts to decrease joint stress, support groups, and pain medications.

Multiple sclerosis (MS) is a demyelinating disease in which the insulating covers of nerve cells in the brain and spinal cord are damaged. This damage disrupts the ability of parts of the nervous system to communicate, resulting in a range of signs and symptoms, including physical, mental, and sometimes psychiatric problems. Specific symptoms can include double vision, blindness in one eye, muscle weakness, trouble with sensation, or trouble with coordination. While the cause is not clear, the underlying mechanism is thought to be either destruction by the immune system or failure of the myelin-producing cells. There is no known cure for multiple sclerosis. Treatments attempt to improve function after an attack and prevent new attacks.

Asthma is a common long-term inflammatory disease of the airways of the lungs. It is characterized by variable and recurring symptoms, reversible airflow obstruction, and bronchospasm. Symptoms include episodes of wheezing, coughing, chest tightness, and shortness of breath. Asthma is thought to be caused by a combination of genetic and environmental factors. Environmental factors include exposure to air pollution and allergens. Asthma is classified according to the frequency of symptoms, forced expiratory volume in one second (FEV1), and peak expiratory flow rate. It may also be classified as atopic or non-atopic, where atopy refers to a predisposition toward developing a type 1 hypersensitivity reaction. There is no cure for asthma. Symptoms can be prevented by avoiding triggers, such as allergens and irritants, and by the use of inhaled corticosteroids. Long-acting beta agonists (LABA) or antileukotriene agents may be used in addition to inhaled corticosteroids if asthma symptoms remain uncontrolled. Treatment of rapidly worsening symptoms is usually with an inhaled short-acting beta-2 agonist such as salbutamol and corticosteroids taken by mouth. In very severe cases, intravenous corticosteroids, magnesium sulfate, and hospitalization may be required.

Chronic obstructive pulmonary disease (COPD) is a type of obstructive lung disease characterized by long-term poor airflow. COPD can include two main conditions, emphysema and chronic bronchitis. In emphysema, the walls between many of the air sacs are damaged. As a result, the air sacs lose their shape and become floppy. This damage also can destroy the walls of the air sacs, leading to fewer and larger air sacs instead of many tiny ones. If this happens, the amount of gas exchange in the lungs is reduced. In chronic bronchitis, the lining of the airways stays constantly irritated and inflamed, and this causes the lining to swell. Lots of thick mucus forms in the airways, making it hard to breathe. There is no known cure for COPD, but the symptoms are treatable and its progression can be delayed.

Pain is a distressing feeling often caused by intense or damaging stimuli, such as stubbing a toe, burning a finger, putting alcohol on a cut, or bumping the “funny bone”. Pain is a complex, subjective phenomenon, defining pain has been a challenge. Pain is also referred to as an unpleasant sensory and emotional experience associated with actual or potential tissue damage. Pain is sometimes regarded as a symptom of an underlying condition, such as inflammation.

### Methods of Manufacture

A variety of known techniques can be utilized in making the polynucleotides, polypeptides, vectors, antigen binding molecules, immune cells, compositions, and the like according to the disclosure.

Prior to the in vitro manipulation or genetic modification of the immune cells described herein, the cells may be obtained from a subject. In some embodiments, the immune cells include T cells. T cells can be obtained from a number of sources, including peripheral blood mononuclear cells (PBMCs), bone marrow, lymph nodes tissue, cord blood, thymus tissue, tissue from a site of infection, ascites, pleural effusion, spleen tissue, and tumors. In certain embodiments, T cells can be obtained from a unit of blood collected from the subject using any number of techniques known to the skilled person, such as FICOLL™ separation. Cells may preferably be obtained from the circulating blood of an individual by apheresis. The apheresis product typically contains lymphocytes, including T cells, monocytes, granulocytes, B cells, other nucleated white blood cells, red blood cells, and platelets. In certain embodiments, the cells collected by apheresis may be washed to remove the plasma fraction, and placed in an appropriate buffer or media for subsequent processing. The cells may be washed with PBS. As will be appreciated, a washing step may be used, such as by using a semiautomated flowthrough centrifuge—for example, the Cobe™ 2991 cell processor, the Baxter CytoMate™, or the like. After washing, the cells may be resuspended in a variety of biocompatible buffers, or other saline solution with or without buffer. In certain embodiments, the undesired components of the apheresis sample may be removed.

In certain embodiments, T cells are isolated from PBMCs by lysing the red blood cells and depleting the monocytes, for example, using centrifugation through a PERCOLL™ gradient. A specific subpopulation of T cells, such as CD28⁺, CD4⁺, CD8⁺, CD45RA⁺, and CD45RO⁺ T cells can be further isolated by positive or negative selection techniques known in the art. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. Flow cytometry and cell sorting may also be used to isolate cell populations of interest for use in the present disclosure.

PBMCs may be used directly for genetic modification with the immune cells (such as CARs or TCRs) using methods as described herein. In certain embodiments, after isolating the PBMCs, T lymphocytes can be further isolated and both cytotoxic and helper T lymphocytes can be sorted into naive, memory, and effector T cell subpopulations either before or after genetic modification and/or expansion.

In some embodiments, CD8+ cells are further sorted into naive, central memory, and effector cells by identifying cell surface antigens that are associated with each of these types of CD8⁺ cells. In some embodiments, the expression of phenotypic markers of central memory T cells include CD45RO, CD62L, CCR7, CD28, CD3, and CD127 and are negative for granzyme B. In some embodiments, central memory T cells are CD45RO⁺, CD62L⁺, CD8⁺ T cells. In some embodiments, effector T cells are negative for CD62L, CCR7, CD28, and CD127, and positive for granzyme B and perforin. In certain embodiments, CD4+ T cells are further sorted into subpopulations. For example, CD4⁺ T helper cells can be sorted into naive, central memory, and effector cells by identifying cell populations that have cell surface antigens.

The immune cells, such as T cells, can be genetically modified following isolation using known methods, or the immune cells can be activated and expanded (or differentiated in the case of progenitors) in vitro prior to being genetically modified. In another embodiment, the immune cells, such as T cells, are genetically modified with the chimeric antigen receptors described herein (e.g., transduced with a viral vector comprising one or more nucleotide sequences encoding a CAR) and then are activated and/or expanded in vitro. Methods for activating and expanding T cells are known in the art and are described, for example, in U.S. Pat. Nos. 6,905,874; 6,867,041; 6,797,514; and PCT WO2012/079000, the contents of which are hereby incorporated by reference in their entirety. Generally, such methods include contacting PBMC or isolated T cells with a stimulatory molecule and a costimulatory molecule, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). One example is The Dynabeads® system, a CD3/CD28 activator/stimulator system for physiological activation of human T cells. In other embodiments, the T cells may be activated and stimulated to proliferate with feeder cells and appropriate antibodies and cytokines using methods such as those described in U.S. Pat. Nos. 6,040,177; 5,827,642; and WO2012129514, the contents of which are hereby incorporated by reference in their entirety.

Certain methods for making the constructs and engineered immune cells of the disclosure are described in PCT application PCT/US15/14520, the contents of which are hereby incorporated by reference in their entirety. Additional methods of making the constructs and cells can be found in U.S. provisional patent application No. 62/244,036 the contents of which are hereby incorporated by reference in their entirety.

It will be appreciated that PBMCs can further include other cytotoxic lymphocytes such as NK cells or NKT cells. An expression vector carrying the coding sequence of a chimeric receptor as disclosed herein can be introduced into a population of human donor T cells, NK cells or NKT cells. Successfully transduced T cells that carry the expression vector can be sorted using flow cytometry to isolate CD3 positive T cells and then further propagated to increase the number of these CAR expressing T cells in addition to cell activation using anti-CD3 antibodies and IL-2 or other methods known in the art as described elsewhere herein. Standard procedures are used for cryopreservation of T cells expressing the CAR for storage and/or preparation for use in a human subject. In one embodiment, the in vitro transduction, culture and/or expansion of T cells are performed in the absence of non-human animal derived products such as fetal calf serum and fetal bovine serum.

For cloning of polynucleotides, the vector may be introduced into a host cell (an isolated host cell) to allow replication of the vector itself and thereby amplify the copies of the polynucleotide contained therein. The cloning vectors may contain sequence components generally include, without limitation, an origin of replication, promoter sequences, transcription initiation sequences, enhancer sequences, and selectable markers. These elements may be selected as appropriate by a person of ordinary skill in the art. For example, the origin of replication may be selected to promote autonomous replication of the vector in the host cell.

In certain embodiments, the present disclosure provides isolated host cells containing the vector provided herein. The host cells containing the vector may be useful in expression or cloning of the polynucleotide contained in the vector. Suitable host cells can include, without limitation, prokaryotic cells, fungal cells, yeast cells, or higher eukaryotic cells such as mammalian cells. Suitable prokaryotic cells for this purpose include, without limitation, eubacteria, such as Gram-negative or Gram-positive organisms, for example, Enterobactehaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

The vector can be introduced to the host cell using any suitable methods known in the art, including, without limitation, DEAE-dextran mediated delivery, calcium phosphate precipitate method, cationic lipids mediated delivery, liposome mediated transfection, electroporation, microprojectile bombardment, receptor-mediated gene delivery, delivery mediated by polylysine, histone, chitosan, and peptides. Standard methods for transfection and transformation of cells for expression of a vector of interest are well known in the art. In a further embodiment, a mixture of different expression vectors can be used in genetically modifying a donor population of immune effector cells wherein each vector encodes a different CAR as disclosed herein. The resulting transduced immune effector cells form a mixed population of engineered cells, with a proportion of the engineered cells expressing more than one different CARs.

In one embodiment, the disclosure provides a method of storing genetically engineered cells expressing CARs or TCRs which target a CLL-1 protein. This involves cryopreserving the immune cells such that the cells remain viable upon thawing. A fraction of the immune cells expressing the CARs can be cryopreserved by methods known in the art to provide a permanent source of such cells for the future treatment of patients afflicted with a malignancy. When needed, the cryopreserved transformed immune cells can be thawed, grown and expanded for more such cells.

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion media can be any isotonic medium formulation, typically normal saline, Normosol™ R (Abbott) or Plasma-Lyte™ A (Baxter), but also 5% dextrose in water or Ringer's lactate can be utilized. The infusion medium can be supplemented with human serum albumin.

Desired treatment amounts of cells in the composition are generally at least 2 cells (for example, at least 1 CD8⁺ central memory T cell and at least 1 CD4⁺ helper T cell subset) or is more typically greater than 10² cells, and up to 10⁶, up to and including 10⁸ or 10⁹ cells and can be more than 10¹⁰ cells. The number of cells will depend upon the desired use for which the composition is intended, and the type of cells included therein. The density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects of the present disclosure, particularly since all the infused cells will be redirected to a particular target antigen (CLL-1), lower numbers of cells, in the range of 10⁶/kilogram (10⁶-10¹¹ per patient) may be administered. CAR treatments may be administered multiple times at dosages within these ranges. The cells may be autologous, allogeneic, or heterologous to the patient undergoing therapy.

The CAR expressing cell populations of the present disclosure may be administered either alone, or as a pharmaceutical composition in combination with diluents and/or with other components such as IL-2 or other cytokines or cell populations. Pharmaceutical compositions of the present disclosure may include a CAR or TCR expressing cell population, such as T cells, as described herein, in combination with one or more pharmaceutically or physiologically acceptable carriers, diluents or excipients. Such compositions may include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for intravenous administration.

The pharmaceutical compositions (solutions, suspensions or the like), may include one or more of the following: sterile diluents such as water for injection, saline solution, preferably physiological saline, Ringer's solution, isotonic sodium chloride, fixed oils such as synthetic mono- or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

It will be appreciated that adverse events may be minimized by transducing the immune cells (containing one or more CARs or TCRs) with a suicide gene. It may also be desired to incorporate an inducible “on” or “accelerator” switch into the immune cells. Suitable techniques include use of inducible caspase-9 (U.S. Appl. 2011/0286980) or a thymidine kinase, before, after or at the same time, as the cells are transduced with the CAR construct of the present disclosure. Additional methods for introducing suicide genes and/or “on” switches include TALENS, zinc fingers, RNAi, siRNA, shRNA, antisense technology, and other techniques known in the art.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

The following examples, including the experiments conducted and results achieved, are provided for illustrative purposes only and are not to be construed as limiting the present disclosure.

### EXAMPLES

### Example 1. Generation of Anti-CLL1 Antigen-Binding Molecules

Murine antibodies against the human CLL1 protein were generated by immunizing 10 female Trianni Flagship mice, aged 6-8 weeks, using Abveris' recommended immunization strategy. The recombinant human CLL1 extracellular domain fused to an Fe fragment (huCLL1-Fe), with a His tag, was used as immunogen.

A single B cell screen using the Berkeley Lights Beacon Platform was performed. Directly preceding B cell screening, plasma B cells were isolated from each immunized mouse chosen for B cell screening. Plasma B cells from each mouse were loaded into an OptoSelect Chip using the Berkeley Lights platform (typically 1 mouse per chip). Typically, 8,000 to 10,000 individual B-cells were loaded and analyzed per chip, though the actual number depended on overlapping variables. Up to 96 plasma B cells were exported and sequenced per mouse screened.

The murine antibodies were screened for binding to huCLL1-Fc and counter-screened against huCLEC12B (Acro Biosystems). Those antibodies binding to huCLL1-Fc without binding to huCLEC12B were selected.

ELISA Validation: Antibodies identified by the Beacon screen were made recombinantly and validated for binding to CLL1. High-binding ELISA plate was coated with muIgG2a monoFc huCLL1 (+) (1 μg/mL) overnight at 4° C. After removal of the antigen, the wells were blocked with 2% BSA in 1×PBS for 1 hour. The BSA was then removed and the primary antibody was added to the wells in 1×PBS solution, incubated at RT for 1 hour. The wells were washed four times with 1×PBS. HRP conjugated secondary antibody was added in blocking solution, and was incubated at RT for 45 minutes. The wells were washed five times with 1×PBS. TMB substrate was added, followed by stop solution. The plates were read at 450 nm.

OD Ranking Analysis: Data from the ELISA were ranked and analyzed (Table 1).

TABLE 1
ELISA ranking

| Concentration in | | muIgG2a monoFc |
|---|---|---|
| well (μg/mL) | Clone | huCLL1 (+) |
| 10.00 | D1D108967-2768 | 2.158 |
| 10.00 | D2AD88607-5141 | 1.998 |
| 6.00 | D1D108967-16071 | 1.967 |
| 10.00 | D2D88607-9226 | 1.747 |
| 10.00 | D2AD89041-3228 | 1.708 |
| 7.00 | D1D108967-13246 | 1.701 |
| 10.00 | D2D88607-7179 | 1.678 |
| 10.00 | D2AD88607-7048 | 1.575 |
| 10.00 | D2D88607-15321 | 1.38 |
| 10.00 | D1D108967-10778 | 0.838 |
| 2.50 | D1D108967-4441 | 0.379 |
| 10.00 | D2AD89041-10444 | 0.205 |
| 7.00 | D2AD88989-6769 | 0.199 |
| 5.50 | D2D88607-14083 | 0.196 |
| 10.00 | D2D88989-16442 | 0.11 |
| 6.00 | D2AD88607-13555 | 0.1 |
| 10.00 | D1D108967-17922 | 0.099 |
| 10.00 | D1D109213-11457 | 0.091 |
| 1.50 | D1D108967-5970 | 0.087 |
| 0.50 | D2D88989-10884 | 0.087 |
| 8.50 | D1D108967-4563 | 0.085 |
| 2.50 | D1D109213-16678 | 0.082 |
| 10.00 | D2D88989-4467 | 0.082 |
| 1.00 | D2AD88607-19838 | 0.081 |
| 0.00 | D2AD88989-5637 | 0.081 |
| 10.00 | D2D88607-15132 | 0.081 |
| 2.50 | D1D109213-15237 | 0.08 |
| 10.00 | D2D88989-14801 | 0.08 |
| 0.00 | D2D89041-13820 | 0.08 |
| 3.00 | D1D109213-7091 | 0.079 |
| 0.50 | D2AD88607-13891 | 0.079 |
| 6.00 | D2AD89041-17592 | 0.079 |
| 3.50 | D2D88607-14076 | 0.079 |
| 10.00 | D2D88607-8979 | 0.079 |
| 10.00 | D2D88607-9010 | 0.079 |
| 4.50 | D1D108967-3113 | 0.077 |
| 0.50 | D2AD88607-16249 | 0.077 |
| 3.00 | D2AD89041-15860 | 0.077 |
| 0.00 | D1D109213-6787 | 0.076 |
| 1.50 | D2AD89041-11043 | 0.076 |
| −1.50 | D2AD89041-3636 | 0.076 |
| 0.00 | D2D88607-12505 | 0.076 |
| 1.00 | D2D88607-990 | 0.076 |
| 8.50 | D1D109213-13036 | 0.075 |
| 0.00 | D2D88607-16518 | 0.075 |
| 0.50 | D2D88989-16363 | 0.075 |
| 6.50 | D1D108967-2461 | 0.074 |
| 5.00 | D2AD88607-13518 | 0.074 |
| 4.00 | D2D88989-6043 | 0.074 |
| 0.00 | D1D109213-4462 | 0.073 |
| 4.50 | D1D109213-5858 | 0.073 |
| 1.50 | D1D109213-6270 | 0.073 |
| 0.00 | D2D89041-11850 | 0.072 |
| −0.50 | D1D109213-16960 | 0.071 |
| 0.50 | D2AD88607-15888 | 0.071 |
| −2.50 | D2D88989-15857 | 0.069 |
| 5.50 | D1D109213-5294 | 0.064 |

Octet Off-Rate Ranking Assay: Alternate binding assay was conducted with the recombinant antibodies confirming specific binding to CLL1 without binding to CLEC12B. Briefly, purified mAbs were individually captured on FAB2G anti-human Fab biosensors. The mAb-captured biosensors were dipped in wells containing antigen and the binding response was measured. Biosensors were later dipped in buffer wells to observe the dissociation of bound antigen from captured mAb and were regenerated and similar binding kinetic assays were performed for subsequent antigens. The binding kinetics of fifteen mAbs were illustrated in Table 2.

TABLE 2
Binding kinetics

| | Binding | muIgG2a | muIgG2a | huIgG1 | huIgG1 |
|---|---|---|---|---|---|
| | Kinetics Exp | huCLL1 | huCLL1 | huCLL1 | huCLL1 |
| Sample ID | mAb ID | K_{D} (M) | t½ (min) | K_{D} (M) | t½ (min) |
| D2AD89041-3228 | mAb_011 | 9.05E−10 | 22.3 | 7.06E−09 | 3.4 |
| D2AD88989-5637 | mAb_046 | 1.55E−09 | 11.2 | 1.03E−08 | 1.9 |
| D2D88607-9226 | mAb_023 | 3.47E−10 | 64.1 | 2.28E−09 | 16.5 |
| D2AD88607-7048 | mAb_042 | 2.98E−09 | 7.7 | 8.11E−08 | 0.3 |
| D1D108967-10778 | mAb_041 | 9.59E−09 | 6.4 | N.B. | N.B. |
| D1D108967-5970 | mAb_040 | 9.82E−09 | 9.8 | N.B. | N.B. |
| D1D108967-4441 | mAb_036 | 9.80E−09 | 9.9 | N.B. | N.B. |
| D2AD88607-5141 | mAb_038 | 7.66E−10 | 54.4 | 1.99E−08 | 2.3 |
| D1D108967-16071 | mAb_014 | 2.93E−10 | 116 | 1.46E−09 | 34.5 |
| D2AD88989-6769 | mAb_050 | 7.33E−09 | 5.3 | 4.92E−07 | 0.2 |
| D2D88607-15321 | mAb_018 | 4.29E−09 | 83.2 | 2.94E−08 | 9.1 |
| D2AD89041-10444 | mAb_049 | 3.46E−09 | 5.4 | 1.12E−06 | 0.0 |
| D2D88607-7179 | mAb_029 | 5.31E−10 | 116 | 3.67E−09 | 17.7 |
| D2D88607-14083 | mAb_017 | 3.74E−10 | 116 | 3.19E−09 | 18.6 |
| D1D108967-13246 | mAb_008 | 2.13E−09 | 116 | 1.82E−08 | 46.9 |

Octet In-Tandem Binning: Competitive binding assay indicating which antibodies compete with each other, thus showing which groups of antibodies bind to the same region of CLL1. Fifteen mAbs were tested:
Step 1: The huCLL1.mIgG2a was captured onto an AMC biosensor.
Step 2: The huCLL1.mIgG2a captured biosensors were later dipped in wells containing 50 μg/mL of first mAb (subsequently referred to as mAb #1) to allow saturation of huCLL1.mIgG2a captured surface.
Step 3: The biosensors were finally dipped in wells containing 50 μg/mL of second mAb (subsequently referred to as mAb #2).
Step 4: Biosensors were regenerated & steps 1-3 were repeated for subsequent mAbs.
Step 5: The in-tandem Octet bin data were analyzed using the Carterra's epitope binning analysis software.
Step 6: The combined dendrograms created based on the cross-competition data were utilized to develop the community plots.
Step 7: Asymmetries represent mAbs that show different competition profile based on the order of addition.

The binning data were represented alternatively by Octet Communities.

Six mAb antibodies were finally selected. The variable region sequences are summarized in Table 3.

TABLE 3
Sequences of anti-CLL-1 antibodies and CLL1

| | | |
|---|---|---|
| Clone | Amino acid sequence (CDRs underlined) | SEQ ID NO: |

mAb_049

| | | |
|---|---|---|
| VL | EIVLTQSPATLSLSPGERATLSCGASQSVTSSYLAWYQQKPGL | 1 |
| | APRLLIYDASNRATGIPDRFSGSGSGTDFTLTISRLEPEDFAV | |
| | YYCQQYGRSLTFGGGTKVEIK | |
| VL CDR1 | QSVTSSY | 3 |
| VL CDR2 | DAS | |
| VL CDR3 | QQYGRSLT | 5 |
| VH | EVQLLESGGGLVQPGGSLRLSCAASGFTFNNYVMAWVRQVPGK | 2 |
| | GLEWVSGISGSGLITSYAASVKDRFTISRDNSNNTVYLQMKSL | |
| | RAEDTAVYFCAKEPHVLLDDYMDVWGKGTTVTVSS | |
| VH CDR1 | GFTFNNYV | 6 |
| VH CDR2 | ISGSGLIT | 7 |
| VH CDR3 | AKEPHVLLDDYMDV | 8 |

mAb_017

| | | |
|---|---|---|
| VL | EIVLTQSPATLSLSPGERATLSCGASQSVSNNYLAWYQQKPGL | 14 |
| | APRLLIYDASSRATGIPDRFSGSGSGTDFTLTISRLEPEDFAV | |
| | YYCQQYGSSHTFGQGTKLEIK | |
| VL CDR1 | QSVSNNY | 16 |
| VL CDR2 | DAS | |
| VL CDR3 | QQYGSSHT | 17 |
| VH | EVQLLESGGDLVQPGGSLRLSCAASGFTFSSYAMNWVRQAPGK | 15 |
| | GLEWVSAISGSGVFTYYADSVKGRFTISRDNSENTLYLQMNSL | |
| | RAEDTAVYYCAKEMQQLIQGDLFDIWGQGTMVTVSS | |
| VH CDR1 | GFTFSSYA | 18 |
| VH CDR2 | ISGSGVFT | 19 |
| VH CDR3 | AKEMQQLIQGDLFDI | 20 |

mAb_023

| | | |
|---|---|---|
| VL | AIQMTQSPSSLSASVGDRVTITCRASQGIRNDLGWYQQKPGKA | 23 |
| | PKLLIYAASSLQSGVPSRFSGSGSGTDFTLTISSLQPEDFATY | |
| | YCLQDYIYPPTFGPGTKLEIK | |
| VL CDR1 | QGIRND | 25 |
| VL CDR2 | AAS | |
| VL CDR3 | LQDYIYPPT | 27 |
| VH | QVQLQQWGAGLLKPSETLSLTCAVYGGSFSGYYWSWIRQPPGK | 24 |
| | GLEWIGEFNHSGGPNYNPSLKSRVTISTDTSNNQFSLKLSSVT | |
| | AADTAVYYCARGTTGNYYYYMDVWGKGTTVTVSS | |
| VH CDR1 | GGSFSGYY | 28 |
| VH CDR2 | FNHSGGP | 29 |
| VH CDR3 | ARGTTGNYYYYMDV | 30 |

mAb_038

| | | |
|---|---|---|
| VL | EIVMTQSPATLSLSPGERATLSCRASQSVRSSYLSWYQQKPGQ | 33 |
| | APRLLIYGTSTRATGIPARFSGSGSGTDFTLTISSLQPEDFAV | |
| | YYCQQDYNLPLTFGGGTKVEIK | |
| VL CDR1 | QSVRSSY | 35 |
| VL CDR2 | GTS | |
| VL CDR3 | QQDYNLPLT | 37 |
| VH | QVQLVESGGGVVQPGRSLRLSCAASGFTFSSYGMHWVRQAPGK | 34 |
| | GLEWVAIISYDGSDKYYADSVKGRFTISRDNSKNTLYLQMNSL | |
| | RAEDTAVYYCARNYNYYMDVWGKGTTVTVSS | |
| VH CDR1 | GFTFSSYG | 38 |
| VH CDR2 | ISYDGSDK | 39 |
| VH CDR3 | ARNYNYYMDV | 40 |

mAb_018

| | | |
|---|---|---|
| VL | EIVLTQSPATLSLSPGERATLSCGASQSVSSSYLAWYQQKPGL | 45 |
| | APRLLIYDASSRATGIPDRFSGSGSGTDFTLTISRLEPEDFAV | |
| | YYCQQYDSSPSTFGQGTKLEIK | |
| VL CDR1 | QSVSSSY | 47 |
| VL CDR2 | DAS | |
| VL CDR3 | QQYDSSPST | 48 |
| VH | EVQLVESGGGLVQPGGSLRLSCAASGFTFSFNDMNWVRQAPGK | 46 |
| | GLEWVSTISGSGGSTYYADSVKGRFTISRDNSKNTLYLQMNSL | |
| | RAEDTAVYYCAVTGLYYYYYMDVWGKGTTVTVSS | |
| VH CDR1 | GFTFSFND | 49 |
| VH CDR2 | ISGSGGST | 50 |
| VH CDR3 | AVTGLYYYYYMDV | 51 |

mAb_042

| | | |
|---|---|---|
| VL | EIVLTQSPDFQSVTPKEKVTITCRASQSIGSGLHWYQQKPDQS | 54 |
| | PKLLIIYASQSISGVPSRFSGSGSGTDFTLTINSLEAEDAAAY | |
| | YCHQSSSLPYTFGQGTKLEIK | |
| VL CDR1 | QSIGSG | 56 |
| VL CDR2 | YAS | |
| VL CDR3 | HQSSSLPYT | 58 |
| VH | EVQLVESGGGLVQPGGSLRLSCAASGFTFSSYDMHWVRQATGK | 55 |
| | GLEWVSAIGTAGDKYYLASVKGRFTISRENAKNSLYLQMNSLR | |
| | AGDTAVYYCVRDSLTGPTVTPYMDVWGKGTTVTVSS | |
| VH CDR1 | GFTFSSYD | 59 |
| VH CDR2 | IGTAGDK | 60 |
| VH CDR3 | VRDSLTGPTVTPYMDV | 61 |
| Human CLL-1 | MSEEVTYADLQFQNSSEMEKIPEIGKFGEKAPPAPSHVWRPAA | 65 |
| | LFLTLLCLLLLIGLGVLASMFHVTLKIEMKKMNKLQNISEELQ | |
| | RNISLQLMSNMNISNKIRNLSTTLQTIATKLCRELYSKEQEHK | |
| | CKPCPRRWIWHKDSCYFLSDDVQTWQESKMACAAQNASLLKIN | |
| Protein | NKNALEFIKSQSRSYDYWLGLSPEEDSTRGMRVDNIINSSAWV | |
| | IRNAPDLNNMYCGYINRLYVQYYHCTYKKRMICEKMANPVQLG | |
| | STYFREA | |

### Example 2. Manufacture of Anti-CLL1 Chimeric Antigen Receptor T Cells

Nine CARs are constructed based on the selected anti-CLL-1 antibodies with structures illustrated in Table 4. The amino acid sequences of relevant components are provided in Table 5, and the full-length CAR constructions are illustrated in Table 6.

TABLE 4
Construction of the CARs

| | | | | | | Co-stimulatory |
|---|---|---|---|---|---|---|
| | | | scFv | | | domain + |
| | | | orienta- | | Trans- | activation |
| | CAR # | mAB # | tion | Hinge | membrane | domain |
| 1 | CAR01 | mAb_049 | VL-VH | CD8 | CD8 | CD28 costim + |
| | | | | | | CD3zeta |
| 2 | CAR02 | mAb_017 | VH-VL | IgG4 | CD28 | CD28 costim + |
| | | | | short | | CD3zeta |
| 3 | CAR03 | mAb_049 | VH-VL | IgG4 | CD28 | CD28 costim + |
| | | | | short | | CD3zeta |
| 4 | CAR04 | mAb_049 | VH-VL | CD28 | CD28 | CD28 costim + |
| | | | | | | CD3zeta |
| 5 | CAR05 | mAb_023 | VH-VL | CD28 | CD28 | CD28 costim + |
| | | | | | | CD3zeta |
| 6 | CAR06 | mAb_038 | VL-VH | CD8 | CD8 | CD28 costim + |
| | | | | | | CD3zeta |
| 7 | CAR07 | mAb_018 | VH-VL | CD28 | CD28 | CD28 costim + |
| | | | | | | CD3zeta |
| 8 | CAR08 | mAb_038 | VH-VL | CD28 | CD28 | CD28 costim + |
| | | | | | | CD3zeta |
| 9 | CAR09 | mAb_042 | VL-VH | IgG4 | CD28 | CD28 costim + |
| | | | | short | | CD3zeta |

TABLE 5
Amino acid sequences of the components of the CAR constructions

| Component | Amino acid sequences | SEQ ID NO: |
|---|---|---|
| Signal peptide | MALPVTALLLPLALLLHAARP | 64 |
| Hinge | | |
| CD8a Hinge | TTTPAPRPPTPAPTIASQPLSLRPEACRPAAGGAVHTRGLD | 78 |
| | FACD | |
| IgG4(S108P)_short | ESKYGPPCPPCP | 69 |
| Hinge | | |
| CD28 Hinge | LDNEKSNGTIIHVKGKHLCPSPLFPGPSKP | 71 |
| Transmembrane | | |
| domain | | |
| CD8a | IYIWAPLAGTCGVLLLSLVITLYC | 77 |
| Transmembrane | | |
| CD28 | FWVLVVVGGVLACYSLLVTVAFIIFWV | 70 |
| Transmembrane | | |
| Costimulatory | | |
| domain | | |
| CD28 costim | RSKRSRLLHSDYMNMTPRRPGPTRKHYQPYAPPRDFAAYRS | 67 |
| Signaling domain | | |
| CD3z AA | RVKFSRSADAPAYQQGQNQLYNELNLGRREEYDVLDKRRGR | 68 |
| | DPEMGGKPRRKNPQEGLYNELQKDKMAEAYSEIGMKGERRR | |
| | GKGHDGLYQGLSTATKDTYDALHMQALPPR | |

TABLE 6
Amino acid sequences of exemplary CAR constructions

| | | |
|---|---|---|
| Element | Amino acid sequences (without signal peptide) | SEQ ID NO: |

1. CAR01 (mAb_049)
aCLL-1(D2AD89041-10444)-VL-3xG4S-VH-AAA-CD8HTM-CD28-CD3z

| | | |
|---|---|---|
| aCLL- | EIVLTQSPATLSLSPGERATLSCGASQSVTSSYLAWYQQKPGLAPR | 10 |
| 1(D2AD89041- | LLIYDASNRATGIPDRFSGSGSGTDFTLTISRLEPEDFAVYYCQQY | |
| 10444)-VL- | GRSLTFGGGTKVEIKGGGGSGGGGSGGGGSEVQLLESGGGLVQPGG | |
| 3xG4S-VH-AAA- | SLRLSCAASGFTFNNYVMAWVRQVPGKGLEWVSGISGSGLITSYAA | |
| CD8HTM(EV)- | SVKDRFTISRDNSNNTVYLQMKSLRAEDTAVYFCAKEPHVLLDDYM | |
| CD28-CD3z CAR | DVWGKGTTVTVSSAAATTTPAPRPPTPAPTIASQPLSLRPEACRPA | |
| | AGGAVHTRGLDFACDIYIWAPLAGTCGVLLLSLVITLYCRSKRSRL | |
| | LHSDYMNMTPRRPGPTRKHYQPYAPPRDFAAYRSRVKFSRSADAPA | |
| | YQQGQNQLYNELNLGRREEYDVLDKRRGRDPEMGGKPRRKNPQEGL | |
| | YNELQKDKMAEAYSEIGMKGERRRGKGHDGLYQGLSTATKDTYDAL | |
| | HMQALPPR | |
| Anti-CLL- | EIVLTQSPATLSLSPGERATLSCGASQSVTSSYLAWYQQKPGLAPR | 9 |
| 1(D2AD89041- | LLIYDASNRATGIPDRFSGSGSGTDFTLTISRLEPEDFAVYYCQQY | |
| 10444)-VL- | GRSLTFGGGTKVEIKGGGGSGGGGSGGGGSEVQLLESGGGLVQPGG | |
| 3xG4S-VH | SLRLSCAASGFTFNNYVMAWVRQVPGKGLEWVSGISGSGLITSYAA | |
| | SVKDRFTISRDNSNNTVYLQMKSLRAEDTAVYFCAKEPHVLLDDYM | |
| | DVWGKGTTVTVSS | |
| AAA linker | AAA | / |
| CD8a Hinge | TTTPAPRPPTPAPTIASQPLSLRPEACRPAAGGAVHTRGLDFACDI | 66 |
| Transmembrane | YIWAPLAGTCGVLLLSLVITLYC | |
| CD28 costim | RSKRSRLLHSDYMNMTPRRPGPTRKHYQPYAPPRDFAAYRS | 67 |
| CD3z | RVKFSRSADAPAYQQGQNQLYNELNLGRREEYDVLDKRRGRDPEMG | 68 |
| | GKPRRKNPQEGLYNELQKDKMAEAYSEIGMKGERRRGKGHDGLYQG | |
| | LSTATKDTYDALHMQALPPR | |

2. CAR02 (mAb_017)
aCLL-1(D2D88607-14083)-VH-3xG4S-VL-AAA-IgG4(S108P)_short-hinge-CD28TM-CD28-CD3z

| | | |
|---|---|---|
| aCLL- | EVQLLESGGDLVQPGGSLRLSCAASGFTFSSYAMNWVRQAPGKGLE | 22 |
| 1(D2D88607- | WVSAISGSGVFTYYADSVKGRFTISRDNSENTLYLQMNSLRAEDTA | |
| 14083)-VH- | VYYCAKEMQQLIQGDLFDIWGQGTMVTVSSGGGGSGGGGSGGGGSE | |
| 3xG4S-VL-AAA- | IVLTQSPATLSLSPGERATLSCGASQSVSNNYLAWYQQKPGLAPRL | |
| IgG4(S108P)_ | LIYDASSRATGIPDRFSGSGSGTDFTLTISRLEPEDFAVYYCQQYG | |
| short-hinge- | SSHTFGQGTKLEIKAAAESKYGPPCPPCPFWVLVVVGGVLACYSLL | |
| CD28TM-CD28- | VTVAFIIFWVRSKRSRLLHSDYMNMTPRRPGPTRKHYQPYAPPRDF | |
| CD3z CAR | AAYRSRVKFSRSADAPAYQQGQNQLYNELNLGRREEYDVLDKRRGR | |
| | DPEMGGKPRRKNPQEGLYNELQKDKMAEAYSEIGMKGERRRGKGHD | |
| | GLYQGLSTATKDTYDALHMQALPPR | |
| Anti-CLL- | EVQLLESGGDLVQPGGSLRLSCAASGFTFSSYAMNWVRQAPGKGLE | 21 |
| 1(D2D88607- | WVSAISGSGVFTYYADSVKGRFTISRDNSENTLYLQMNSLRAEDTA | |
| 14083)-VH- | VYYCAKEMQQLIQGDLFDIWGQGTMVTVSSGGGGSGGGGSGGGGSE | |
| 3xG4S-VL | IVLTQSPATLSLSPGERATLSCGASQSVSNNYLAWYQQKPGLAPRL | |
| | LIYDASSRATGIPDRFSGSGSGTDFTLTISRLEPEDFAVYYCQQYG | |
| | SSHTFGQGTKLEIK | |
| AAA linker | AAA | / |
| IgG4(S108P)_ | ESKYGPPCPPCP | 69 |
| short Hinge | | |
| CD28 | FWVLVVVGGVLACYSLLVTVAFIIFWV | 70 |
| Transmembrane | | |
| CD28 costim | RSKRSRLLHSDYMNMTPRRPGPTRKHYQPYAPPRDFAAYRS | 67 |
| CD3z | RVKFSRSADAPAYQQGQNQLYNELNLGRREEYDVLDKRRGRDPEMG | 68 |
| | GKPRRKNPQEGLYNELQKDKMAEAYSEIGMKGERRRGKGHDGLYQG | |
| | LSTATKDTYDALHMQALPPR | |

3. CAR03 (mAb_049)
aCLL-1(D2AD89041-10444)-VH-3xG4S-VL-AAA-IgG4(S108P)_short-Hinge-CD28TM-CD28-CD3z

| | | |
|---|---|---|
| aCLL- | EVQLLESGGGLVQPGGSLRLSCAASGFTFNNYVMAWVRQVPGKGLE | 12 |
| 1(D2AD89041- | WVSGISGSGLITSYAASVKDRFTISRDNSNNTVYLQMKSLRAEDTA | |
| 10444)-VH- | VYFCAKEPHVLLDDYMDVWGKGTTVTVSSGGGGSGGGGSGGGGSEI | |
| 3xG4S-VL-AAA- | VLTQSPATLSLSPGERATLSCGASQSVTSSYLAWYQQKPGLAPRLL | |
| IgG4(S108P)_ | IYDASNRATGIPDRFSGSGSGTDFTLTISRLEPEDFAVYYCQQYGR | |
| short-Hinge- | SLTFGGGTKVEIKAAAESKYGPPCPPCPFWVLVVVGGVLACYSLLV | |
| CD28TM-CD28- | TVAFIIFWVRSKRSRLLHSDYMNMTPRRPGPTRKHYQPYAPPRDFA | |
| CD3z | AYRSRVKFSRSADAPAYQQGQNQLYNELNLGRREEYDVLDKRRGRD | |
| | PEMGGKPRRKNPQEGLYNELQKDKMAEAYSEIGMKGERRRGKGHDG | |
| | LYQGLSTATKDTYDALHMQALPPR | |
| Anti-CLL- | EVQLLESGGGLVQPGGSLRLSCAASGFTFNNYVMAWVRQVPGKGLE | 11 |
| 1(D2AD89041- | WVSGISGSGLITSYAASVKDRFTISRDNSNNTVYLQMKSLRAEDTA | |
| 10444)-VH- | VYFCAKEPHVLLDDYMDVWGKGTTVTVSSGGGGSGGGGSGGGGSEI | |
| 3xG4S-VL | VLTQSPATLSLSPGERATLSCGASQSVTSSYLAWYQQKPGLAPRLL | |
| | IYDASNRATGIPDRFSGSGSGTDFTLTISRLEPEDFAVYYCQQYGR | |
| | SLTFGGGTKVEIK | |
| AAA linker | AAA | 1 |
| IgG4(S108P)_ | ESKYGPPCPPCP | 69 |
| short Hinge | | |
| CD28 | FWVLVVVGGVLACYSLLVTVAFIIFWV | 70 |
| Transmembrane | | |
| CD28 costim | RSKRSRLLHSDYMNMTPRRPGPTRKHYQPYAPPRDFAAYRS | 67 |
| CD3z | RVKFSRSADAPAYQQGQNQLYNELNLGRREEYDVLDKRRGRDPEMG | 68 |
| | GKPRRKNPQEGLYNELQKDKMAEAYSEIGMKGERRRGKGHDGLYQG | |
| | LSTATKDTYDALHMQALPPR | |

4. CAR04 (mAb_049)
aCLL-1(D2AD89041-10444)-VH-3xG4S-VL-AAA-CD28-Hinge-CD28_TM-CD28-CD3z

| | | |
|---|---|---|
| aCLL- | EVQLLESGGGLVQPGGSLRLSCAASGFTFNNYVMAWVRQVPGKGLE | 13 |
| 1(D2AD89041- | WVSGISGSGLITSYAASVKDRFTISRDNSNNTVYLQMKSLRAEDTA | |
| 10444)-VH- | VYFCAKEPHVLLDDYMDVWGKGTTVTVSSGGGGSGGGGSGGGGSEI | |
| 3xG4S-VL-AAA- | VLTQSPATLSLSPGERATLSCGASQSVTSSYLAWYQQKPGLAPRLL | |
| CD28-Hinge- | IYDASNRATGIPDRFSGSGSGTDFTLTISRLEPEDFAVYYCQQYGR | |
| CD28_TM-CD28- | SLTFGGGTKVEIKAAALDNEKSNGTIIHVKGKHLCPSPLFPGPSKP | |
| CD3z | FWVLVVVGGVLACYSLLVTVAFIIFWVRSKRSRLLHSDYMNMTPRR | |
| | PGPTRKHYQPYAPPRDFAAYRSRVKFSRSADAPAYQQGQNQLYNEL | |
| | NLGRREEYDVLDKRRGRDPEMGGKPRRKNPQEGLYNELQKDKMAEA | |
| | YSEIGMKGERRRGKGHDGLYQGLSTATKDTYDALHMQALPPR | |
| Anti-CLL- | EVQLLESGGGLVQPGGSLRLSCAASGFTFNNYVMAWVRQVPGKGLE | 11 |
| 1(D2AD89041- | WVSGISGSGLITSYAASVKDRFTISRDNSNNTVYLQMKSLRAEDTA | |
| 10444)-VH- | VYFCAKEPHVLLDDYMDVWGKGTTVTVSSGGGGSGGGGSGGGGSEI | |
| 3xG4S-VL AA | VLTQSPATLSLSPGERATLSCGASQSVTSSYLAWYQQKPGLAPRLL | |
| | IYDASNRATGIPDRFSGSGSGTDFTLTISRLEPEDFAVYYCQQYGR | |
| | SLTFGGGTKVEIK | |
| AAA linker | AAA | / |
| CD28 Hinge | LDNEKSNGTIIHVKGKHLCPSPLFPGPSKP | 71 |
| CD28 | FWVLVVVGGVLACYSLLVTVAFIIFWV | 70 |
| Transmembrane | | |
| CD28 costim | RSKRSRLLHSDYMNMTPRRPGPTRKHYQPYAPPRDFAAYRS | 67 |
| CD3z | RVKFSRSADAPAYQQGQNQLYNELNLGRREEYDVLDKRRGRDPEMG | 68 |
| | GKPRRKNPQEGLYNELQKDKMAEAYSEIGMKGERRRGKGHDGLYQG | |
| | LSTATKDTYDALHMQALPPR | |

5. CAR05 (mAb_023)
aCLL-1(D2D88607-9226)-VH-3xG4S-VL-AAA-CD28-Hinge-CD28_TM-CD28-CD3z

| | | |
|---|---|---|
| aCLL- | QVQLQQWGAGLLKPSETLSLTCAVYGGSFSGYYWSWIRQPPGKGLE | 32 |
| 1(D2D88607- | WIGEFNHSGGPNYNPSLKSRVTISTDTSNNQFSLKLSSVTAADTAV | |
| 9226)-VH- | YYCARGTTGNYYYYMDVWGKGTTVTVSSGGGGSGGGGSGGGGSAIQ | |
| 3xG4S-VL-AAA- | MTQSPSSLSASVGDRVTITCRASQGIRNDLGWYQQKPGKAPKLLIY | |
| CD28-Hinge- | AASSLQSGVPSRFSGSGSGTDFTLTISSLQPEDFATYYCLQDYIYP | |
| CD28_TM-CD28- | PTFGPGTKLEIKAAALDNEKSNGTIIHVKGKHLCPSPLFPGPSKPF | |
| CD3z | WVLVVVGGVLACYSLLVTVAFIIFWVRSKRSRLLHSDYMNMTPRRP | |
| | GPTRKHYQPYAPPRDFAAYRSRVKFSRSADAPAYQQGQNQLYNELN | |
| | LGRREEYDVLDKRRGRDPEMGGKPRRKNPQEGLYNELQKDKMAEAY | |
| | SEIGMKGERRRGKGHDGLYQGLSTATKDTYDALHMQALPPR | |
| Anti-CLL- | QVQLQQWGAGLLKPSETLSLTCAVYGGSFSGYYWSWIRQPPGKGLE | 31 |
| 1(D2D88607- | WIGEFNHSGGPNYNPSLKSRVTISTDTSNNQFSLKLSSVTAADTAV | |
| 9226)-VH- | YYCARGTTGNYYYYMDVWGKGTTVTVSSGGGGSGGGGSGGGGSAIQ | |
| 3xG4S-VL | MTQSPSSLSASVGDRVTITCRASQGIRNDLGWYQQKPGKAPKLLIY | |
| | AASSLQSGVPSRFSGSGSGTDFTLTISSLQPEDFATYYCLQDYIYP | |
| | PTFGPGTKLEIK | |
| AAA linker | AAA | / |
| CD28 Hinge | LDNEKSNGTIIHVKGKHLCPSPLFPGPSKP | 71 |
| CD28 | FWVLVVVGGVLACYSLLVTVAFIIFWV | 70 |
| Transmembrane | | |
| CD28 costim | RSKRSRLLHSDYMNMTPRRPGPTRKHYQPYAPPRDFAAYRS | 67 |
| CD3z | RVKFSRSADAPAYQQGQNQLYNELNLGRREEYDVLDKRRGRDPEMG | 68 |
| | GKPRRKNPQEGLYNELQKDKMAEAYSEIGMKGERRRGKGHDGLYQG | |
| | LSTATKDTYDALHMQALPPR | |

6. CAR06 (mAb_038)
aCLL-1(D2AD88607-5141)-VL-3xG4S-VH-AAA-CD8HTM-CD28-CD3z

| | | |
|---|---|---|
| aCLL- | EIVMTQSPATLSLSPGERATLSCRASQSVRSSYLSWYQQKPGQAPR | 42 |
| 1(D2AD88607- | LLIYGTSTRATGIPARFSGSGSGTDFTLTISSLQPEDFAVYYCQQD | |
| 5141)-VL-3xG4S- | YNLPLTFGGGTKVEIKGGGGSGGGGSGGGGSQVQLVESGGGVVQPG | |
| VH-AAA- | RSLRLSCAASGFTFSSYGMHWVRQAPGKGLEWVAIISYDGSDKYYA | |
| CD8HTM-CD28- | DSVKGRFTISRDNSKNTLYLQMNSLRAEDTAVYYCARNYNYYMDVW | |
| CD3z | GKGTTVTVSSAAATTTPAPRPPTPAPTIASQPLSLRPEACRPAAGG | |
| | AVHTRGLDFACDIYIWAPLAGTCGVLLLSLVITLYCRSKRSRLLHS | |
| | DYMNMTPRRPGPTRKHYQPYAPPRDFAAYRSRVKFSRSADAPAYQQ | |
| | GQNQLYNELNLGRREEYDVLDKRRGRDPEMGGKPRRKNPQEGLYNE | |
| | LQKDKMAEAYSEIGMKGERRRGKGHDGLYQGLSTATKDTYDALHMQ | |
| | ALPPR | |
| Anti-CLL- | EIVMTQSPATLSLSPGERATLSCRASQSVRSSYLSWYQQKPGQAPR | 41 |
| 1(D2AD88607- | LLIYGTSTRATGIPARFSGSGSGTDFTLTISSLQPEDFAVYYCQQD | |
| 5141)-VL-3xG4S- | YNLPLTFGGGTKVEIKGGGGSGGGGSGGGGSQVQLVESGGGVVQPG | |
| VH | RSLRLSCAASGFTFSSYGMHWVRQAPGKGLEWVAIISYDGSDKYYA | |
| | DSVKGRFTISRDNSKNTLYLQMNSLRAEDTAVYYCARNYNYYMDVW | |
| | GKGTTVTVSS | |
| AAA linker | AAA | / |
| CD8a Hinge-TM | TTTPAPRPPTPAPTIASQPLSLRPEACRPAAGGAVHTRGLDFACDI | 66 |
| | YIWAPLAGTCGVLLLSLVITLYC | |
| CD28 costim | RSKRSRLLHSDYMNMTPRRPGPTRKHYQPYAPPRDFAAYRS | 67 |
| CD3z | RVKFSRSADAPAYQQGQNQLYNELNLGRREEYDVLDKRRGRDPEMG | 68 |
| | GKPRRKNPQEGLYNELQKDKMAEAYSEIGMKGERRRGKGHDGLYQG | |
| | LSTATKDTYDALHMQALPPR | |

7. CAR07 (mAb_018)
aCLL-1(D2D88607-15321)-VH-3xG4S-VL-AAA-CD28-Hinge-TM-CD28-CD3z

| | | |
|---|---|---|
| aCLL- | EVQLVESGGGLVQPGGSLRLSCAASGFTFSFNDMNWVRQAPGKGLE | 53 |
| 1(D2D88607- | WVSTISGSGGSTYYADSVKGRFTISRDNSKNTLYLQMNSLRAEDTA | |
| 15321)-VH- | VYYCAVTGLYYYYYMDVWGKGTTVTVSSGGGGSGGGGSGGGGSEIV | |
| 3xG4S-VL-AAA- | LTQSPATLSLSPGERATLSCGASQSVSSSYLAWYQQKPGLAPRLLI | |
| CD28-Hinge-TM- | YDASSRATGIPDRFSGSGSGTDFTLTISRLEPEDFAVYYCQQYDSS | |
| CD28-CD3z CAR | PSTFGQGTKLEIKAAALDNEKSNGTIIHVKGKHLCPSPLFPGPSKP | |
| | FWVLVVVGGVLACYSLLVTVAFIIFWVRSKRSRLLHSDYMNMTPRR | |
| | PGPTRKHYQPYAPPRDFAAYRSRVKFSRSADAPAYQQGQNQLYNEL | |
| | NLGRREEYDVLDKRRGRDPEMGGKPRRKNPQEGLYNELQKDKMAEA | |
| | YSEIGMKGERRRGKGHDGLYQGLSTATKDTYDALHMQALPPR | |
| Anti-CLL- | EVQLVESGGGLVQPGGSLRLSCAASGFTFSFNDMNWVRQAPGKGLE | 52 |
| 1(D2D88607- | WVSTISGSGGSTYYADSVKGRFTISRDNSKNTLYLQMNSLRAEDTA | |
| 15321)-VH- | VYYCAVTGLYYYYYMDVWGKGTTVTVSSGGGGSGGGGSGGGGSEIV | |
| 3xG4S-VL | LTQSPATLSLSPGERATLSCGASQSVSSSYLAWYQQKPGLAPRLLI | |
| | YDASSRATGIPDRFSGSGSGTDFTLTISRLEPEDFAVYYCQQYDSS | |
| | PSTFGQGTKLEIK | |
| AAA linker | AAA | / |
| CD28 Hinge | LDNEKSNGTIIHVKGKHLCPSPLFPGPSKP | 71 |
| CD28 TM | FWVLVVVGGVLACYSLLVTVAFIIFWV | 70 |
| CD28 costim | RSKRSRLLHSDYMNMTPRRPGPTRKHYQPYAPPRDFAAYRS | 67 |
| CD3z | RVKFSRSADAPAYQQGQNQLYNELNLGRREEYDVLDKRRGRDPEMG | 68 |
| | GKPRRKNPQEGLYNELQKDKMAEAYSEIGMKGERRRGKGHDGLYQG | |
| | LSTATKDTYDALHMQALPPR | |

8. CAR08 (mAb_038)
aCLL-1(D2AD88607-5141)-VH-3xG4S-VL-AAA-CD28-Hinge-TM-CD28-CD3z

| | | |
|---|---|---|
| aCLL- | QVQLVESGGGVVQPGRSLRLSCAASGFTFSSYGMHWVRQAPGKGLE | 44 |
| 1(D2AD88607- | WVAIISYDGSDKYYADSVKGRFTISRDNSKNTLYLQMNSLRAEDTA | |
| 5141)-VH- | VYYCARNYNYYMDVWGKGTTVTVSSGGGGSGGGGSGGGGSEIVMTQ | |
| 3xG4S-VL-AAA- | SPATLSLSPGERATLSCRASQSVRSSYLSWYQQKPGQAPRLLIYGT | |
| CD28-Hinge-TM- | STRATGIPARFSGSGSGTDFTLTISSLQPEDFAVYYCQQDYNLPLT | |
| CD28-CD3z | FGGGTKVEIKAAALDNEKSNGTIIHVKGKHLCPSPLFPGPSKPFWV | |
| | LVVVGGVLACYSLLVTVAFIIFWVRSKRSRLLHSDYMNMTPRRPGP | |
| | TRKHYQPYAPPRDFAAYRSRVKFSRSADAPAYQQGQNQLYNELNLG | |
| | RREEYDVLDKRRGRDPEMGGKPRRKNPQEGLYNELQKDKMAEAYSE | |
| | IGMKGERRRGKGHDGLYQGLSTATKDTYDALHMQALPPR | |
| Anti-CLL- | QVQLVESGGGVVQPGRSLRLSCAASGFTFSSYGMHWVRQAPGKGLE | 43 |
| 1(D2AD88607- | WVAIISYDGSDKYYADSVKGRFTISRDNSKNTLYLQMNSLRAEDTA | |
| 5141)-VH- | VYYCARNYNYYMDVWGKGTTVTVSSGGGGSGGGGSGGGGSEIVMTQ | |
| 3xG4S-VL | SPATLSLSPGERATLSCRASQSVRSSYLSWYQQKPGQAPRLLIYGT | |
| | STRATGIPARFSGSGSGTDFTLTISSLQPEDFAVYYCQQDYNLPLT | |
| | FGGGTKVEIK | |
| AAA linker | AAA | / |
| CD28 Hinge | LDNEKSNGTIIHVKGKHLCPSPLFPGPSKP | 71 |
| CD28 TM | FWVLVVVGGVLACYSLLVTVAFIIFWV | 70 |
| CD28 costim | RSKRSRLLHSDYMNMTPRRPGPTRKHYQPYAPPRDFAAYRS | 67 |
| CD3z | RVKFSRSADAPAYQQGQNQLYNELNLGRREEYDVLDKRRGRDPEMG | 68 |
| | GKPRRKNPQEGLYNELQKDKMAEAYSEIGMKGERRRGKGHDGLYQG | |
| | LSTATKDTYDALHMQALPPR | |

9. CAR09 (mAb_042)
aCLL-1(D2AD88607-7048)-VL-3xG4S-VH-AAA-IgG4(S108P)_short-CD28HTM-CD28-CD3z

| | | |
|---|---|---|
| aCLL- | EIVLTQSPDFQSVTPKEKVTITCRASQSIGSGLHWYQQKPDQSPKL | 63 |
| 1(D2AD88607- | LIIYASQSISGVPSRFSGSGSGTDFTLTINSLEAEDAAAYYCHQSS | |
| 7048)-VL-3xG4S- | SLPYTFGQGTKLEIKGGGGSGGGGSGGGGSEVQLVESGGGLVQPGG | |
| VH-AAA- | SLRLSCAASGFTFSSYDMHWVRQATGKGLEWVSAIGTAGDKYYLAS | |
| IgG4(S108P)_sho | VKGRFTISRENAKNSLYLQMNSLRAGDTAVYYCVRDSLTGPTVTPY | |
| rt-CD28HTM- | MDVWGKGTTVTVSSAAAESKYGPPCPPCPFWVLVVVGGVLACYSLL | |
| CD28-CD3z | VTVAFIIFWVRSKRSRLLHSDYMNMTPRRPGPTRKHYQPYAPPRDE | |
| | AAYRSRVKFSRSADAPAYQQGQNQLYNELNLGRREEYDVLDKRRGR | |
| | DPEMGGKPRRKNPQEGLYNELQKDKMAEAYSEIGMKGERRRGKGHD | |
| | GLYQGLSTATKDTYDALHMQALPPR | |
| Anti-CLL- | EIVLTQSPDFQSVTPKEKVTITCRASQSIGSGLHWYQQKPDQSPKL | 62 |
| 1(D2AD88607- | LIIYASQSISGVPSRFSGSGSGTDFTLTINSLEAEDAAAYYCHQSS | |
| 7048)-VL-3xG4S- | SLPYTFGQGTKLEIKGGGGSGGGGSGGGGSEVQLVESGGGLVQPGG | |
| VH | SLRLSCAASGFTFSSYDMHWVRQATGKGLEWVSAIGTAGDKYYLAS | |
| | VKGRFTISRENAKNSLYLQMNSLRAGDTAVYYCVRDSLTGPTVTPY | |
| | MDVWGKGTTVTVSS | |
| AAA linker | AAA | / |
| IgG4(S108P)_sho | ESKYGPPCPPCP | 69 |
| rt Hinge | | |
| CD28 TM | FWVLVVVGGVLACYSLLVTVAFIIFWV | 70 |
| CD28 costim | RSKRSRLLHSDYMNMTPRRPGPTRKHYQPYAPPRDFAAYRS | 67 |
| CD3z | RVKFSRSADAPAYQQGQNQLYNELNLGRREEYDVLDKRRGRDPEMG | 68 |
| | GKPRRKNPQEGLYNELQKDKMAEAYSEIGMKGERRRGKGHDGLYQG | |
| | LSTATKDTYDALHMQALPPR | |

The chimeric antigen receptors (CARs) were prepared with donor T cells following the protocol as summarized as below.

| | |
|---|---|
| Process | Small Scale: G-Rex |
| Donor | Donor 01 & Donor 02 |
| IL2 Concentration | 300 |
| (IU/ml) | |
| MOI | 1 |
| Activation Vessel | 6-well plate |
| Expansion Vessel | 24 well G-rex |
| Day 0 | Coat each well with 3 mL of OKT3 to achieve final concentration of 1.23 ug/mL |
| | Thaw and activate cells 6E6 cells/well with media, IL-2, and soluble CD28 (1.0 |
| | ug/mL). |
| Day 1 | Transduce at 2E6 cells/mL with IL-2, virus supernatant, and media. Final volume |
| | should be 4 ml in a 6-well plate with final IL2 concentration of 150 IU/mL. |
| | No transduction (NTD): Add media + IL2. Final volume should be 4 mL in a 6- |
| | well plate with final IL2 concentration of 150 IU/mL. |
| Day 3 | Collect cells from wells, wash and count. Keep replicates separate. Transfer each |
| | replicate into a well of a 24-well G-Rex |
| Day 6 | Perform 75% media exchange. Add IL-2. Count cells. Pull samples for |
| | transduction check and measure of vector copy number (VCN). |
| Day 8* | Harvest, Count, Phenotype, VCN lysates - keep replicates separate. Pool |
| | replicates, freeze down. |
| | If continuing culture to Day 10, perform 75% media exchange and add IL-2. |

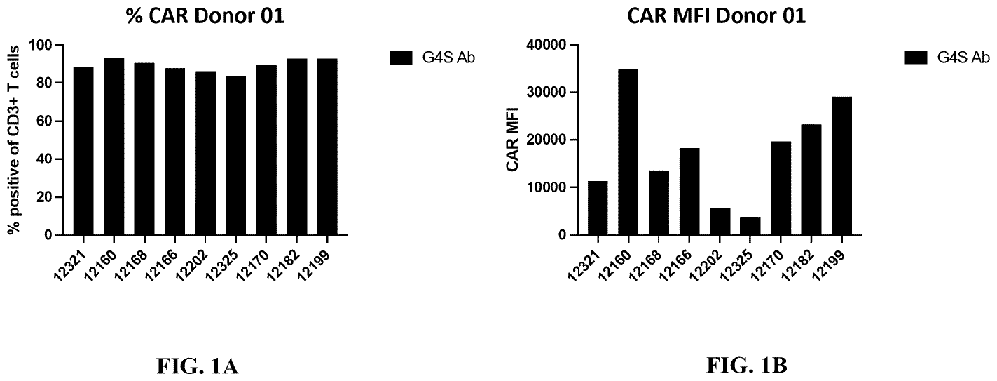

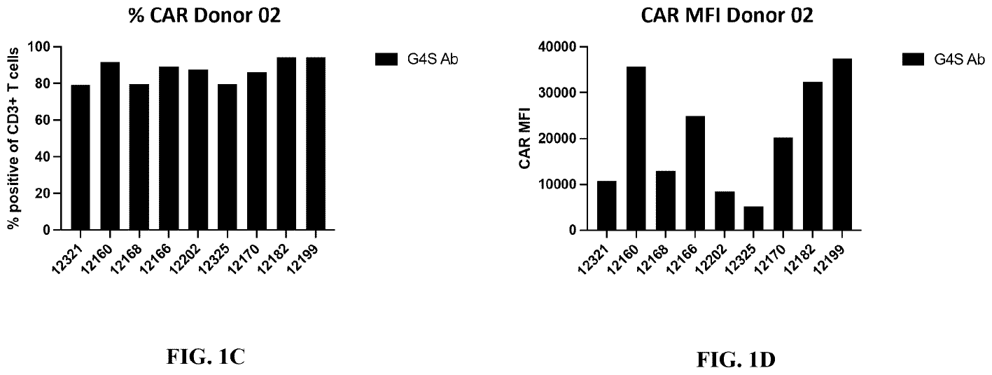

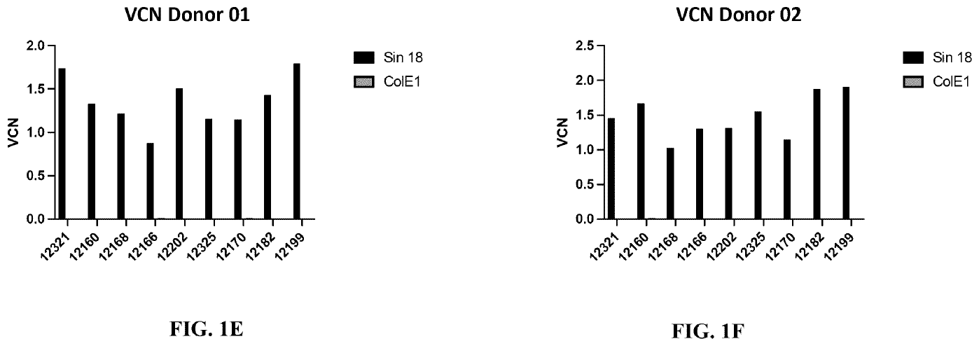

The expression of the anti-CLL-1 CARs in the donor T cells of Donor 01 (FIG. 1A and FIG. 1B) and Donor 02 (FIG. 1C and FIG. 1D) was detected using an anti-G4S antibody (“G4S” disclosed as SEQ ID NO: 74). The VCN in the donor cells were also collected (FIG. 1E and FIG. 1F). In both donor cells, the CAR expression was quite high.

### Example 3. Characterization of the CAR-Expressing T Cells

### Memory T Cell Phenotype Expressing the CARs

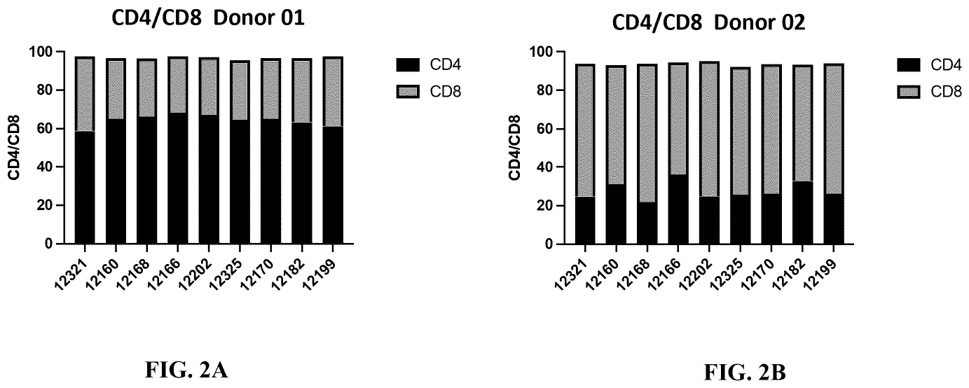

The CAR expression phenotype in the memory T cells has been tested. As shown in FIG. 2A and FIG. 2B, the CD4+/CD8+ ratios of the memory T cells expressing the CARs were different in Donor 01 (FIG. 2A) and Donor 02 (FIG. 2B).

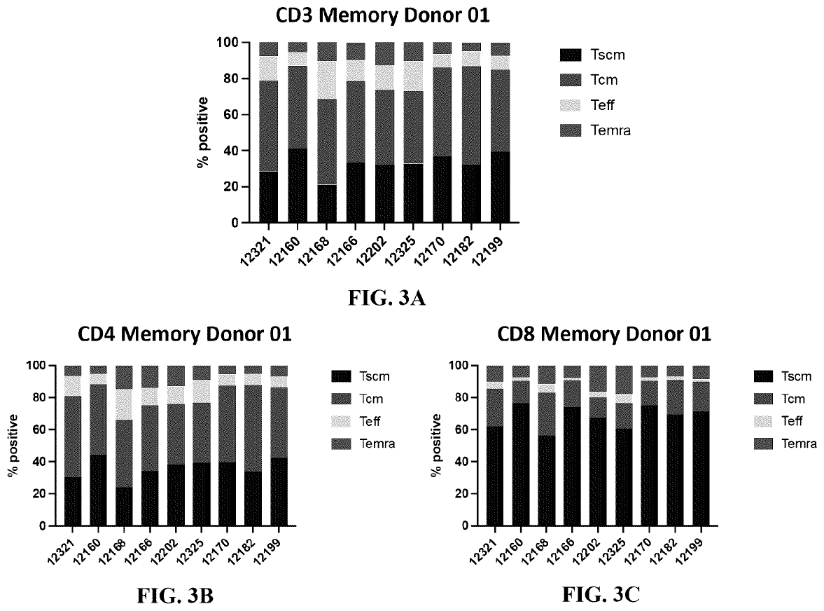

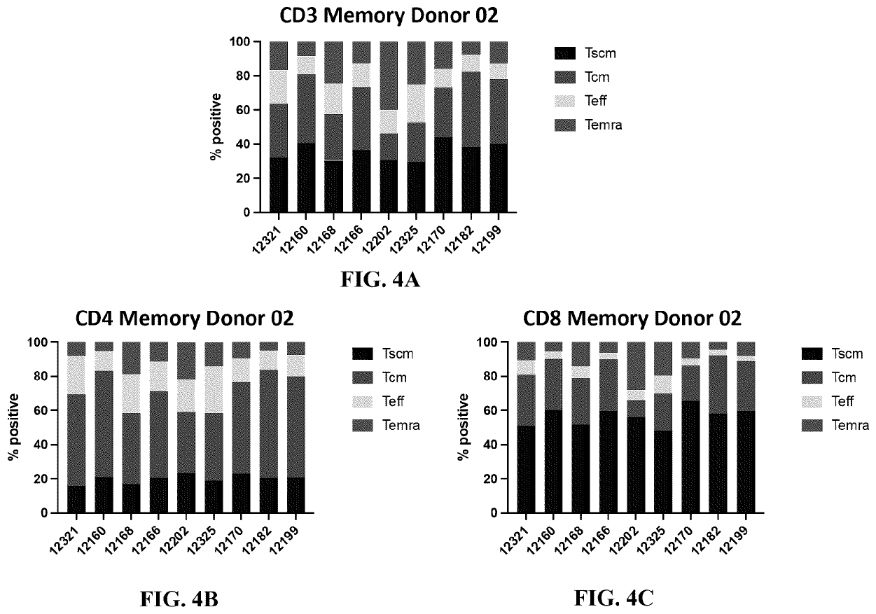

Based on CD45RA and CCR7 expression, the percentage of stem cell-like memory T cells (Tscm), central memory (Tcm), effector T cell (Teff), and effector memory cells re-expressing CD45RA (Temra) T-cells that expressed the CARs were measured in the memory T cells from Donor 01 (FIG. 3A CD3+ T cells, FIG. 3B CD4+ T cells, FIG. 3C CD8+ T cells) and Donor 02 (FIG. 4A CD3+ T cells, FIG. 4B CD4+ T cells, FIG. 4C CD8+ T cells).

### Cytotoxicity of CAR-T Cells In Vitro

The in vitro cytotoxic activity of the CAR-T cells was measured. After 24 hr and 96 hr of incubation, the killing rate (cytotoxicity) of the CAR-T cells to different target cells (MV4-11, Kasumi-1 and K562) was measured at effector versus target (E:T) ratios of 1:1 and 1:1

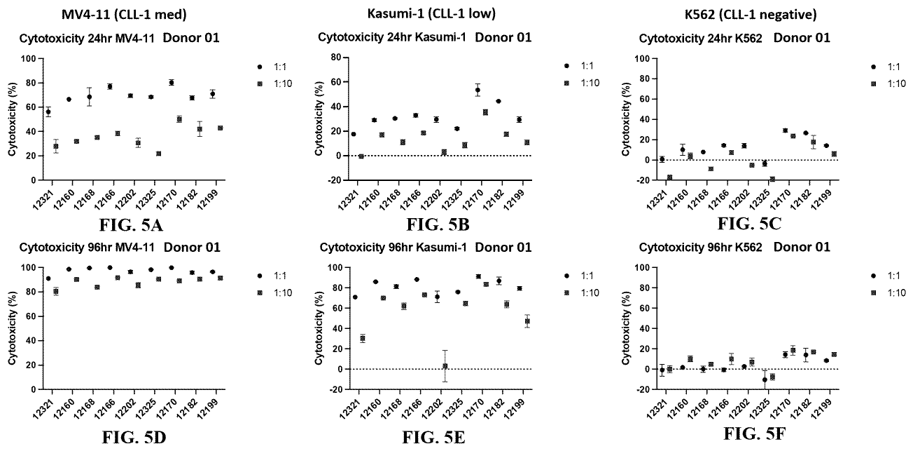

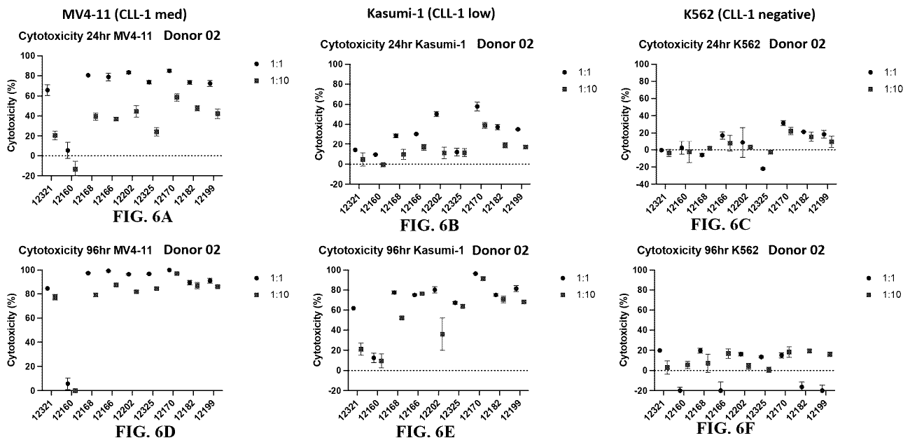

0, respectively (as seen in the graphs in FIGS. 5A-5F and FIGS. 6A-6F). MV4-11 and Kasumi-1 are human AML cell lines positive in CLL1 (high and low CLL1 expression, respectively), and K562 is a CLL1− human erythroleukemic cell line. All CARs, in both donor cells, exhibited potent cytotoxic activities against MV4-11 and Kasumi-1 but not against CLL1-K562.

### T Cell Activation by CAR-T Cells In Vitro

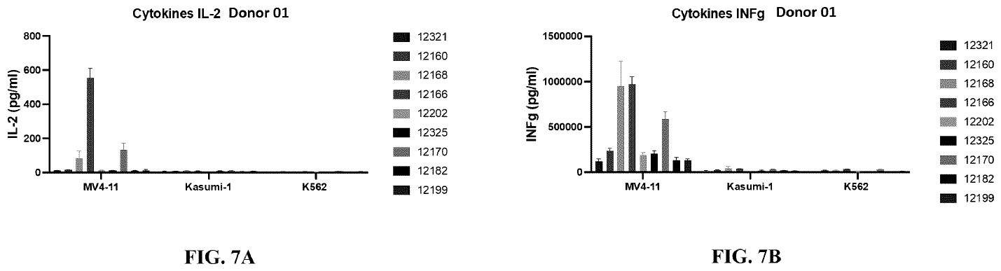

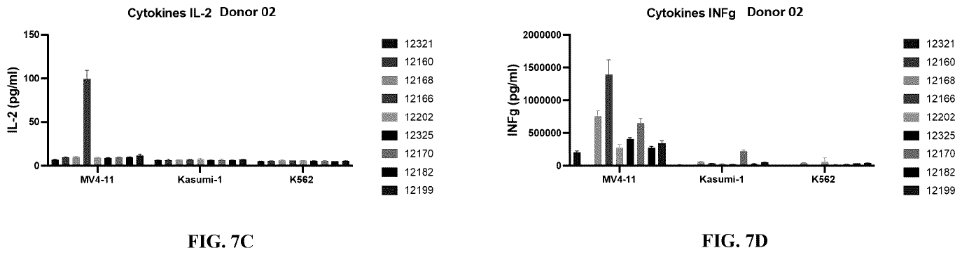

The efficacy of the CAR structures on T cell activation was tested by assessing the IL-2 and IFN-gamma released by CAR T cells when they responded to the tumor cells MV4-11, Kasumi-1 and K562 (FIG. 7A, FIG. 7B, FIG. 7C and FIG. 7D).

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. An antibody or antigen-binding fragment thereof having specificity to the human C-type lectin-like molecule-1 (CLL-1) protein, comprising a heavy chain variable region (VH) comprising a VH CDR1, a VH CDR2 and a VH CDR3, and a light chain variable region (VL) comprising a VL CDR1, a VL CDR2 and a VL CDR3, wherein the VH CDR1, VH CDR2, VH CDR3, VL CDR1, VL CDR2 and VL CDR3 comprise, respectively,
(i) the VH CDR1, VH CDR2 and VH CDR3 sequences of SEQ ID NO:2 and the VL CDR1, VL CDR2 and VL CDR3 sequences of SEQ ID NO:1;
(ii) the VH CDR1, VH CDR2 and VH CDR3 sequences of SEQ ID NO:15 and the VL CDR1, VL CDR2 and VL CDR3 sequences of SEQ ID NO:14;
(iii) the VH CDR1, VH CDR2 and VH CDR3 sequences of SEQ ID NO:24 and the VL CDR1, VL CDR2 and VL CDR3 sequences of SEQ ID NO:23;
(iv) the VH CDR1, VH CDR2 and VH CDR3 sequences of SEQ ID NO:34 and the VL CDR1, VL CDR2 and VL CDR3 sequences of SEQ ID NO:33;
(v) the VH CDR1, VH CDR2 and VH CDR3 sequences of SEQ ID NO:46 and the VL CDR1, VL CDR2 and VL CDR3 sequences of SEQ ID NO:45; or
(vi) the VH CDR1, VH CDR2 and VH CDR3 sequences of SEQ ID NO:55 and the VL CDR1, VL CDR2 and VL CDR3 sequences of SEQ ID NO:54.

2. The antibody or antigen-binding fragment thereof of claim 1, wherein the VH CDR1, VH CDR2, VH CDR3, VL CDR1, VL CDR2 and VL CDR3 comprise, respectively, the sequences of
(i)
GFTFNNYV, ISGSGLIT, AKEPHVLLDDYMDV, QSVTSSY, DAS,
and QQYGRSLT;
(ii)
GFTFSSYA, ISGSGVFT, AKEMQQLIQGDLFDI, QSVSNNY, DAS,
and QQYGSSHT;
(iii)
GGSFSGYY, FNHSGGP, ARGTTGNYYYYMDV, QGIRND, AAS,
and LQDYIYPPT;
(iv)
GFTFSSYG, ISYDGSDK, ARNYNYYMDV, QSVRSSY, GTS,
and QQDYNLPLT;
(v)
GFTFSFND, ISGSGGST, AVTGLYYYYYMDV, QSVSSSY, DAS,
and QQYDSSPST;
or
(vi)
GFTFSSYD, IGTAGDK, VRDSLTGPTVTPYMDV, QSIGSG, YAS,
and HQSSSLPYT.

3. The antibody or antigen-binding fragment thereof of claim 2, wherein the VH CDR1, VH CDR2, VH CDR3, VL CDR1, VL CDR2 and VL CDR3 comprise, respectively, the sequences of GFTFNNYV, ISGSGLIT, AKEPHVLLDDYMDV, QSVTSSY, DAS, and QQYGRSLT.

4. The antibody or antigen-binding fragment thereof of claim 3, wherein the VH comprises the sequence of SEQ ID NO:2 and the VL comprises the sequence of SEQ ID NO:1.

5. The antibody or antigen-binding fragment thereof of claim 2, wherein the VH CDR1, VH CDR2, VH CDR3, VL CDR1, VL CDR2 and VL CDR3 comprise, respectively, the sequences of GFTFSSYA, ISGSGVFT, AKEMQQLIQGDLFDI, QSVSNNY, DAS, and QQYGSSHT.

6. The antibody or antigen-binding fragment thereof of claim 5, wherein the VH comprises the sequence of SEQ ID NO:15 and the VL comprises the sequence of SEQ ID NO:14.

7. The antibody or antigen-binding fragment thereof of claim 2, wherein the VH CDR1, VH CDR2, VH CDR3, VL CDR1, VL CDR2 and VL CDR3 comprise, respectively, the sequences of GGSFSGYY, FNHSGGP, ARGTTGNYYYYMDV, QGIRND, AAS, and LQDYIYPPT.

8. The antibody or antigen-binding fragment thereof of claim 7, wherein the VH comprises the sequence of SEQ ID NO:24 and the VL comprises the sequence of SEQ ID NO:23.

9. The antibody or antigen-binding fragment thereof of claim 2, wherein the VH CDR1, VH CDR2, VH CDR3, VL CDR1, VL CDR2 and VL CDR3 comprise, respectively, the sequences of GFTFSSYG, ISYDGSDK, ARNYNYYMDV, QSVRSSY, GTS, and QQDYNLPLT.

10. The antibody or antigen-binding fragment thereof of claim 9, wherein the VH comprises the sequence of SEQ ID NO:34 and the VL comprises the sequence of SEQ ID NO:33.

11. The antibody or antigen-binding fragment thereof of claim 2, wherein the VH CDR1, VH CDR2, VH CDR3, VL CDR1, VL CDR2 and VL CDR3 comprise, respectively, the sequences of GFTFSFND, ISGSGGST, AVTGLYYYYYMDV, QSVSSSY, DAS, and QQYDSSPST.

12. The antibody or antigen-binding fragment thereof of claim 11, wherein the VH comprises the sequence of SEQ ID NO:46 and the VL comprises the sequence of SEQ ID NO:45.

13. The antibody or antigen-binding fragment thereof of claim 2, wherein the VH CDR1, VH CDR2, VH CDR3, VL CDR1, VL CDR2 and VL CDR3 comprise, respectively, the sequences of GFTFSSYD, IGTAGDK, VRDSLTGPTVTPYMDV, QSIGSG, YAS, and HQSSSLPYT.

14. The antibody or antigen-binding fragment thereof of claim 13, wherein the VH comprises the sequence of SEQ ID NO:55 and the VL comprises the sequence of SEQ ID NO:54.

15. A chimeric antigen receptor (CAR) having specificity to the human CLL-1 protein, comprising an antigen-binding fragment of claim 1, a transmembrane domain, and an intracellular activating domain, and
wherein the antigen-binding fragment is a single chain fragment (scFv).

16.-32. (canceled)

33. A nucleic acid encoding the antibody or antigen-binding fragment thereof of claim 1.

34. A vector comprising the nucleic acid of claim 33.

35. A composition comprising the antibody or antigen-binding fragment thereof of claim 1, and a pharmaceutically acceptable carrier.

36. A cell encoding or expressing the antibody or antigen-binding fragment thereof of any claim 1, optionally wherein the cell is an immune cell, optionally wherein the cell is a T cell.

37. (canceled)

38. A method of generating an engineered cell, the method comprising transfecting or transducing a cell with a nucleic acid of claim 33.

39. A method of treating cancer or an autoimmune disease in a subject in need thereof, the method comprising administering to the subject one or more cells that encode or comprise the antibody or antigen-binding fragment thereof of claim 1.

40. A method of inducing an immune response in a subject, the method comprising administering to the subject one or more cells that encode or comprise the antibody or antigen-binding fragment thereof of claim 1.

41.-44. (canceled)


SEQUENCE LISTING (78 sequences)

SEQ ID NO: 1 - protein, 107 aa, synthetic construct
EIVLTQSPATLSLSPGERATLSCGASQSVTSSYLAWYQQKPGLAPRLLIYDASNRATGIPDRFSGSGSGTDFTLTISRLEPEDFAVYYCQQYGRSLTFGGGTKVEIK

SEQ ID NO: 2 - protein, 121 aa, synthetic construct
EVQLLESGGGLVQPGGSLRLSCAASGFTFNNYVMAWVRQVPGKGLEWVSGISGSGLITSYAASVKDRFTISRDNSNNTVYLQMKSLRAEDTAVYFCAKEPHVLLDDYMDVWGKGTTVTVSS

SEQ ID NO: 3 - protein, 7 aa, synthetic construct
QSVTSSY

SEQ ID NO: 4 - skipped

SEQ ID NO: 5 - protein, 8 aa, synthetic construct
QQYGRSLT

SEQ ID NO: 6 - protein, 8 aa, synthetic construct
GFTFNNYV

SEQ ID NO: 7 - protein, 8 aa, synthetic construct
ISGSGLIT

SEQ ID NO: 8 - protein, 14 aa, synthetic construct
AKEPHVLLDDYMDV

SEQ ID NO: 9 - protein, 243 aa, synthetic construct
EIVLTQSPATLSLSPGERATLSCGASQSVTSSYLAWYQQKPGLAPRLLIYDASNRATGIPDRFSGSGSGTDFTLTISRLEPEDFAVYYCQQYGRSLTFGGGTKVEIKGGGGSGGGGSGGGGSEVQLLESGGGLVQPGGSLRLSCAASGFTFNNYVMAWVRQVPGKGLEWVSGISGSGLITSYAASVKDRFTISRDNSNNTVYLQMKSLRAEDTAVYFCAKEPHVLLDDYMDVWGKGTTVTVSS

SEQ ID NO: 10 - protein, 468 aa, synthetic construct
EIVLTQSPATLSLSPGERATLSCGASQSVTSSYLAWYQQKPGLAPRLLIYDASNRATGIPDRFSGSGSGTDFTLTISRLEPEDFAVYYCQQYGRSLTFGGGTKVEIKGGGGSGGGGSGGGGSEVQLLESGGGLVQPGGSLRLSCAASGFTFNNYVMAWVRQVPGKGLEWVSGISGSGLITSYAASVKDRFTISRDNSNNTVYLQMKSLRAEDTAVYFCAKEPHVLLDDYMDVWGKGTTVTVSSAAATTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 243 aa, synthetic construct
EVQLLESGGGLVQPGGSLRLSCAASGFTFNNYVMAWVRQVPGKGLEWVSGISGSGLITSYAASVKDRFTISRDNSNNTVYLQMKSLRAEDTAVYFCAKEPHVLLDDYMDVWGKGTTVTVSSGGGGSGGGGSGGGGSEIVLTQSPATLSLSPGERATLSCGASQSVTSSYLAWYQQKPGLAPRLLIYDASNRATGIPDRFSGSGSGTDFTLTISRLEPEDFAVYYCQQYGRSLTFGGGTKVEIK

SEQ ID NO: 12 - protein, 438 aa, synthetic construct
EVQLLESGGGLVQPGGSLRLSCAASGFTFNNYVMAWVRQVPGKGLEWVSGISGSGLITSYAASVKDRFTISRDNSNNTVYLQMKSLRAEDTAVYFCAKEPHVLLDDYMDVWGKGTTVTVSSGGGGSGGGGSGGGGSEIVLTQSPATLSLSPGERATLSCGASQSVTSSYLAWYQQKPGLAPRLLIYDASNRATGIPDRFSGSGSGTDFTLTISRLEPEDFAVYYCQQYGRSLTFGGGTKVEIKAAAESKYGPPCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 456 aa, synthetic construct
EVQLLESGGGLVQPGGSLRLSCAASGFTFNNYVMAWVRQVPGKGLEWVSGISGSGLITSYAASVKDRFTISRDNSNNTVYLQMKSLRAEDTAVYFCAKEPHVLLDDYMDVWGKGTTVTVSSGGGGSGGGGSGGGGSEIVLTQSPATLSLSPGERATLSCGASQSVTSSYLAWYQQKPGLAPRLLIYDASNRATGIPDRFSGSGSGTDFTLTISRLEPEDFAVYYCQQYGRSLTFGGGTKVEIKAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 107 aa, synthetic construct
EIVLTQSPATLSLSPGERATLSCGASQSVSNNYLAWYQQKPGLAPRLLIYDASSRATGIPDRFSGSGSGTDFTLTISRLEPEDFAVYYCQQYGSSHTFGQGTKLEIK

SEQ ID NO: 15 - protein, 122 aa, synthetic construct
EVQLLESGGDLVQPGGSLRLSCAASGFTFSSYAMNWVRQAPGKGLEWVSAISGSGVFTYYADSVKGRFTISRDNSENTLYLQMNSLRAEDTAVYYCAKEMQQLIQGDLFDIWGQGTMVTVSS

SEQ ID NO: 16 - protein, 7 aa, synthetic construct
QSVSNNY

SEQ ID NO: 17 - protein, 8 aa, synthetic construct
QQYGSSHT

SEQ ID NO: 18 - protein, 8 aa, synthetic construct
GFTFSSYA

SEQ ID NO: 19 - protein, 8 aa, synthetic construct
ISGSGVFT

SEQ ID NO: 20 - protein, 15 aa, synthetic construct
AKEMQQLIQGDLFDI

SEQ ID NO: 21 - protein, 244 aa, synthetic construct
EVQLLESGGDLVQPGGSLRLSCAASGFTFSSYAMNWVRQAPGKGLEWVSAISGSGVFTYYADSVKGRFTISRDNSENTLYLQMNSLRAEDTAVYYCAKEMQQLIQGDLFDIWGQGTMVTVSSGGGGSGGGGSGGGGSEIVLTQSPATLSLSPGERATLSCGASQSVSNNYLAWYQQKPGLAPRLLIYDASSRATGIPDRFSGSGSGTDFTLTISRLEPEDFAVYYCQQYGSSHTFGQGTKLEIK

SEQ ID NO: 22 - protein, 439 aa, synthetic construct
EVQLLESGGDLVQPGGSLRLSCAASGFTFSSYAMNWVRQAPGKGLEWVSAISGSGVFTYYADSVKGRFTISRDNSENTLYLQMNSLRAEDTAVYYCAKEMQQLIQGDLFDIWGQGTMVTVSSGGGGSGGGGSGGGGSEIVLTQSPATLSLSPGERATLSCGASQSVSNNYLAWYQQKPGLAPRLLIYDASSRATGIPDRFSGSGSGTDFTLTISRLEPEDFAVYYCQQYGSSHTFGQGTKLEIKAAAESKYGPPCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 107 aa, synthetic construct
AIQMTQSPSSLSASVGDRVTITCRASQGIRNDLGWYQQKPGKAPKLLIYAASSLQSGVPSRFSGSGSGTDFTLTISSLQPEDFATYYCLQDYIYPPTFGPGTKLEIK

SEQ ID NO: 24 - protein, 120 aa, synthetic construct
QVQLQQWGAGLLKPSETLSLTCAVYGGSFSGYYWSWIRQPPGKGLEWIGEFNHSGGPNYNPSLKSRVTISTDTSNNQFSLKLSSVTAADTAVYYCARGTTGNYYYYMDVWGKGTTVTVSS

SEQ ID NO: 25 - protein, 6 aa, synthetic construct
QGIRND

SEQ ID NO: 26 - skipped

SEQ ID NO: 27 - protein, 9 aa, synthetic construct
LQDYIYPPT

SEQ ID NO: 28 - protein, 8 aa, synthetic construct
GGSFSGYY

SEQ ID NO: 29 - protein, 7 aa, synthetic construct
FNHSGGP

SEQ ID NO: 30 - protein, 14 aa, synthetic construct
ARGTTGNYYYYMDV

SEQ ID NO: 31 - protein, 242 aa, synthetic construct
QVQLQQWGAGLLKPSETLSLTCAVYGGSFSGYYWSWIRQPPGKGLEWIGEFNHSGGPNYNPSLKSRVTISTDTSNNQFSLKLSSVTAADTAVYYCARGTTGNYYYYMDVWGKGTTVTVSSGGGGSGGGGSGGGGSAIQMTQSPSSLSASVGDRVTITCRASQGIRNDLGWYQQKPGKAPKLLIYAASSLQSGVPSRFSGSGSGTDFTLTISSLQPEDFATYYCLQDYIYPPTFGPGTKLEIK

SEQ ID NO: 32 - protein, 455 aa, synthetic construct
QVQLQQWGAGLLKPSETLSLTCAVYGGSFSGYYWSWIRQPPGKGLEWIGEFNHSGGPNYNPSLKSRVTISTDTSNNQFSLKLSSVTAADTAVYYCARGTTGNYYYYMDVWGKGTTVTVSSGGGGSGGGGSGGGGSAIQMTQSPSSLSASVGDRVTITCRASQGIRNDLGWYQQKPGKAPKLLIYAASSLQSGVPSRFSGSGSGTDFTLTISSLQPEDFATYYCLQDYIYPPTFGPGTKLEIKAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 108 aa, synthetic construct
EIVMTQSPATLSLSPGERATLSCRASQSVRSSYLSWYQQKPGQAPRLLIYGTSTRATGIPARFSGSGSGTDFTLTISSLQPEDFAVYYCQQDYNLPLTFGGGTKVEIK

SEQ ID NO: 34 - protein, 117 aa, synthetic construct
QVQLVESGGGVVQPGRSLRLSCAASGFTFSSYGMHWVRQAPGKGLEWVAIISYDGSDKYYADSVKGRFTISRDNSKNTLYLQMNSLRAEDTAVYYCARNYNYYMDVWGKGTTVTVSS

SEQ ID NO: 35 - protein, 7 aa, synthetic construct
QSVRSSY

SEQ ID NO: 36 - skipped

SEQ ID NO: 37 - protein, 9 aa, synthetic construct
QQDYNLPLT

SEQ ID NO: 38 - protein, 8 aa, synthetic construct
GFTFSSYG

SEQ ID NO: 39 - protein, 8 aa, synthetic construct
ISYDGSDK

SEQ ID NO: 40 - protein, 10 aa, synthetic construct
ARNYNYYMDV

SEQ ID NO: 41 - protein, 240 aa, synthetic construct
EIVMTQSPATLSLSPGERATLSCRASQSVRSSYLSWYQQKPGQAPRLLIYGTSTRATGIPARFSGSGSGTDFTLTISSLQPEDFAVYYCQQDYNLPLTFGGGTKVEIKGGGGSGGGGSGGGGSQVQLVESGGGVVQPGRSLRLSCAASGFTFSSYGMHWVRQAPGKGLEWVAIISYDGSDKYYADSVKGRFTISRDNSKNTLYLQMNSLRAEDTAVYYCARNYNYYMDVWGKGTTVTVSS

SEQ ID NO: 42 - protein, 465 aa, synthetic construct
EIVMTQSPATLSLSPGERATLSCRASQSVRSSYLSWYQQKPGQAPRLLIYGTSTRATGIPARFSGSGSGTDFTLTISSLQPEDFAVYYCQQDYNLPLTFGGGTKVEIKGGGGSGGGGSGGGGSQVQLVESGGGVVQPGRSLRLSCAASGFTFSSYGMHWVRQAPGKGLEWVAIISYDGSDKYYADSVKGRFTISRDNSKNTLYLQMNSLRAEDTAVYYCARNYNYYMDVWGKGTTVTVSSAAATTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 240 aa, synthetic construct
QVQLVESGGGVVQPGRSLRLSCAASGFTFSSYGMHWVRQAPGKGLEWVAIISYDGSDKYYADSVKGRFTISRDNSKNTLYLQMNSLRAEDTAVYYCARNYNYYMDVWGKGTTVTVSSGGGGSGGGGSGGGGSEIVMTQSPATLSLSPGERATLSCRASQSVRSSYLSWYQQKPGQAPRLLIYGTSTRATGIPARFSGSGSGTDFTLTISSLQPEDFAVYYCQQDYNLPLTFGGGTKVEIK

SEQ ID NO: 44 - protein, 453 aa, synthetic construct
QVQLVESGGGVVQPGRSLRLSCAASGFTFSSYGMHWVRQAPGKGLEWVAIISYDGSDKYYADSVKGRFTISRDNSKNTLYLQMNSLRAEDTAVYYCARNYNYYMDVWGKGTTVTVSSGGGGSGGGGSGGGGSEIVMTQSPATLSLSPGERATLSCRASQSVRSSYLSWYQQKPGQAPRLLIYGTSTRATGIPARFSGSGSGTDFTLTISSLQPEDFAVYYCQQDYNLPLTFGGGTKVEIKAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 108 aa, synthetic construct
EIVLTQSPATLSLSPGERATLSCGASQSVSSSYLAWYQQKPGLAPRLLIYDASSRATGIPDRFSGSGSGTDFTLTISRLEPEDFAVYYCQQYDSSPSTFGQGTKLEIK

SEQ ID NO: 46 - protein, 120 aa, synthetic construct
EVQLVESGGGLVQPGGSLRLSCAASGFTFSFNDMNWVRQAPGKGLEWVSTISGSGGSTYYADSVKGRFTISRDNSKNTLYLQMNSLRAEDTAVYYCAVTGLYYYYYMDVWGKGTTVTVSS

SEQ ID NO: 47 - protein, 7 aa, synthetic construct
QSVSSSY

SEQ ID NO: 48 - protein, 9 aa, synthetic construct
QQYDSSPST

SEQ ID NO: 49 - protein, 8 aa, synthetic construct
GFTFSFND

SEQ ID NO: 50 - protein, 8 aa, synthetic construct
ISGSGGST

SEQ ID NO: 51 - protein, 13 aa, synthetic construct
AVTGLYYYYYMDV

SEQ ID NO: 52 - protein, 243 aa, synthetic construct
EVQLVESGGGLVQPGGSLRLSCAASGFTFSFNDMNWVRQAPGKGLEWVSTISGSGGSTYYADSVKGRFTISRDNSKNTLYLQMNSLRAEDTAVYYCAVTGLYYYYYMDVWGKGTTVTVSSGGGGSGGGGSGGGGSEIVLTQSPATLSLSPGERATLSCGASQSVSSSYLAWYQQKPGLAPRLLIYDASSRATGIPDRFSGSGSGTDFTLTISRLEPEDFAVYYCQQYDSSPSTFGQGTKLEIK

SEQ ID NO: 53 - protein, 456 aa, synthetic construct
EVQLVESGGGLVQPGGSLRLSCAASGFTFSFNDMNWVRQAPGKGLEWVSTISGSGGSTYYADSVKGRFTISRDNSKNTLYLQMNSLRAEDTAVYYCAVTGLYYYYYMDVWGKGTTVTVSSGGGGSGGGGSGGGGSEIVLTQSPATLSLSPGERATLSCGASQSVSSSYLAWYQQKPGLAPRLLIYDASSRATGIPDRFSGSGSGTDFTLTISRLEPEDFAVYYCQQYDSSPSTFGQGTKLEIKAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 107 aa, synthetic construct
EIVLTQSPDFQSVTPKEKVTITCRASQSIGSGLHWYQQKPDQSPKLLIIYASQSISGVPSRFSGSGSGTDFTLTINSLEAEDAAAYYCHQSSSLPYTFGQGTKLEIK

SEQ ID NO: 55 - protein, 122 aa, synthetic construct
EVQLVESGGGLVQPGGSLRLSCAASGFTFSSYDMHWVRQATGKGLEWVSAIGTAGDKYYLASVKGRFTISRENAKNSLYLQMNSLRAGDTAVYYCVRDSLTGPTVTPYMDVWGKGTTVTVSS

SEQ ID NO: 56 - protein, 6 aa, synthetic construct
QSIGSG

SEQ ID NO: 57 - skipped

SEQ ID NO: 58 - protein, 9 aa, synthetic construct
HQSSSLPYT

SEQ ID NO: 59 - protein, 8 aa, synthetic construct
GFTFSSYD

SEQ ID NO: 60 - protein, 7 aa, synthetic construct
IGTAGDK

SEQ ID NO: 61 - protein, 16 aa, synthetic construct
VRDSLTGPTVTPYMDV

SEQ ID NO: 62 - protein, 244 aa, synthetic construct
EIVLTQSPDFQSVTPKEKVTITCRASQSIGSGLHWYQQKPDQSPKLLIIYASQSISGVPSRFSGSGSGTDFTLTINSLEAEDAAAYYCHQSSSLPYTFGQGTKLEIKGGGGSGGGGSGGGGSEVQLVESGGGLVQPGGSLRLSCAASGFTFSSYDMHWVRQATGKGLEWVSAIGTAGDKYYLASVKGRFTISRENAKNSLYLQMNSLRAGDTAVYYCVRDSLTGPTVTPYMDVWGKGTTVTVSS

SEQ ID NO: 63 - protein, 439 aa, synthetic construct
EIVLTQSPDFQSVTPKEKVTITCRASQSIGSGLHWYQQKPDQSPKLLIIYASQSISGVPSRFSGSGSGTDFTLTINSLEAEDAAAYYCHQSSSLPYTFGQGTKLEIKGGGGSGGGGSGGGGSEVQLVESGGGLVQPGGSLRLSCAASGFTFSSYDMHWVRQATGKGLEWVSAIGTAGDKYYLASVKGRFTISRENAKNSLYLQMNSLRAGDTAVYYCVRDSLTGPTVTPYMDVWGKGTTVTVSSAAAESKYGPPCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 21 aa, synthetic construct
MALPVTALLLPLALLLHAARP

SEQ ID NO: 65 - protein, 265 aa, Homo sapiens
MSEEVTYADLQFQNSSEMEKIPEIGKFGEKAPPAPSHVWRPAALFLTLLCLLLLIGLGVLASMFHVTLKIEMKKMNKLQNISEELQRNISLQLMSNMNISNKIRNLSTTLQTIATKLCRELYSKEQEHKCKPCPRRWIWHKDSCYFLSDDVQTWQESKMACAAQNASLLKINNKNALEFIKSQSRSYDYWLGLSPEEDSTRGMRVDNIINSSAWVIRNAPDLNNMYCGYINRLYVQYYHCTYKKRMICEKMANPVQLGSTYFREA

SEQ ID NO: 66 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 67 - protein, 41 aa, synthetic construct
RSKRSRLLHSDYMNMTPRRPGPTRKHYQPYAPPRDFAAYRS

SEQ ID NO: 68 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 12 aa, synthetic construct
ESKYGPPCPPCP

SEQ ID NO: 70 - protein, 27 aa, synthetic construct
FWVLVVVGGVLACYSLLVTVAFIIFWV

SEQ ID NO: 71 - protein, 30 aa, synthetic construct
LDNEKSNGTIIHVKGKHLCPSPLFPGPSKP

SEQ ID NO: 72 - protein, 4 aa, synthetic construct
GGGS

SEQ ID NO: 73 - protein, 15 aa, synthetic construct
GGGGSGGGGSGGGGS

SEQ ID NO: 74 - protein, 5 aa, synthetic construct
GGGGS

SEQ ID NO: 75 - protein, 5 aa, synthetic construct
GSGGS

SEQ ID NO: 76 - protein, 20 aa, synthetic construct
GGGGSGGGGSGGGGSGGGGS

SEQ ID NO: 77 - protein, 24 aa, synthetic construct
IYIWAPLAGTCGVLLLSLVITLYC

SEQ ID NO: 78 - protein, 45 aa, synthetic construct
TTTPAPRPPTPAPTIASQPLSLRPEACRPAAGGAVHTRGLDFACD